(19) United States
(12) Patent Application Publication
(10) Pub. No.: US 2005/0132438 A1
(43) Pub. Date: Jun. 16, 2005

(54) Novel monocotylednous plant genes and uses thereof

(76) Inventors: Helen Ziaohui Wang, Cary, NC (US); John Manuel Salmeron, Hillsborough, NC (US); Michael Gregory Willits, Apex, NC (US); Kay Ann Lawton, Raleigh, NC (US)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(21) Appl. No.: 10/220,665

(22) PCT Filed: Mar. 5, 2001
(86) PCT No.: PCT/EP01/02463

Related U.S. Application Data
(60) Provisional application No. 60/228,798, filed on Mar. 6, 2000.

(51) Int. Cl. C12Q 1/68; C07H 21/04; C12N 9/24; A01H 1/00; C12N 15/82; C12N 5/04

(52) U.S. Cl. 800/279; 435/6; 435/69.1; 435/200; 435/419; 435/468; 536/23.2

(57) ABSTRACT

Homologues of the Arabidopsis NIM1 gene, which is involved in the signal transduction cascade leading to systemic acquired resistance (SAR), are isolated from monocotyledonous crops such as Triticum aestivum (wheat) and Oryza sativa (rice). The invention further concerns transformation vectors and processes for expressing the monocotyledonous NIM1 homologues in transgenic plants to increase SAR gene expression and enhance broad spectrum disease resistance

The present invention relates to broad-spectrum disease resistance in plants, including the phenomenon of systemic acquired resistance (SAR). More particularly, the present invention relates to the identification, isolation and characterization of monocotyledonous homologues of the NIM1 gene involved in the signal transduction cascade leading to systemic acquired resistance in plants.

Plants are constantly challenged by a wide variety of pathogenic organisms including viruses, bacteria, fungi, and nematodes. Crop plants are particularly vulnerable because they are usually grown as genetically-uniform monocultures; when disease strikes, losses can be severe. However, most plants have their own innate mechanisms of defense against pathogenic organisms. Natural variation for resistance to plant pathogens has been identified by plant breeders and pathologists and bred into many crop plants. These natural disease resistance genes often provide high levels of resistance to or immunity against pathogens.

Systemic acquired resistance (SAR) is one component of the complex system plants use to defend themselves from pathogens (Hunt and Ryals, 1996; Ryals et al., 1996). See also, U.S. Pat. No. 5,614,395. SAR is a particularly important aspect of plant-pathogen responses because it is a pathogen-inducible, systemic resistance against a broad spectrum of infectious agents, including viruses, bacteria, and fungi. When the SAR signal transduction pathway is blocked, plants become more susceptible to pathogens that normally cause disease, and they also become susceptible to some infectious agents that would not normally cause disease (Gaffney et al., 1993; Delaney et al., 1994; Delaney et al., 1995; Delaney, 1997; Bi et al., 1995; Mauch-Mani and Slusarenko, 1996). These observations indicate that the SAR signal transduction pathway is critical for maintaining plant health.

Conceptually, the SAR response can be divided into two phases. In the initiation phase, a pathogen infection is recognized, and a signal is released that travels through the phloem to distant tissues. This systemic signal is perceived by target cells, which react by expression of both SAR genes and disease resistance. The maintenance phase of SAR refers to the period of time, from weeks up to the entire life of the plant, during which the plant is in a quasi steady state, and disease resistance is maintained (Ryals et al., 1996).

Salicylic acid (SA) accumulation appears to be required for SAR signal transduction. Plants that cannot accumulate SA due to treatment with specific inhibitors, epigenetic repression of phenylalanine ammonia-lyase, or transgenic expression of salicylate hydroxylase, which specifically degrades SA, also cannot induce either SAR gene expression or disease resistance (Gaffney et al., 1993; Delaney et al., 1994; Mauch-Mani and Slusarenko, 1996; Maher et al., 1994; Pallas et al., 1996). Although it has been suggested that SA might serve as the systemic signal, this is currently controversial and, to date, all that is known for certain is that if SA cannot accumulate, then SAR signal transduction is blocked (Pallas et al., 1996; Shulaev et al., 1995; Vernooij et al., 1994).

Recently, Arabidopsis has emerged as a model system to study SAR (Uknes et al., 1992; Uknes et al., 1993; Cameron et al., 1994; Mauch-Mani and Slusarenko, 1994; Dempsey and Klessig, 1995). It has been demonstrated that SAR can be activated in Arabidopsis by both pathogens and chemicals, such as SA, 2,6-dichloroisonicotinic acid (INA) and benzo(1,2,3)thiadiazole-7-carbothioic acid S-methyl ester (BTH) (Uknes et al., 1992; Vernooij et al., 1995; Lawton et al., 1996). Following treatment with either INA or BTH or pathogen infection, at least three pathogenesis-related (PR) protein genes, namely, PR-1, PR-2, and PR-5 are coordinately induced concomitant with the onset of resistance (Uknes et al., 1992, 1993). In tobacco, the best characterized species, treatment with a pathogen or an immunization compound induces the expression of at least nine sets of genes (Ward et al., 1991). Transgenic disease-resistant plants have been created by transforming plants with various SAR genes (U.S. Pat. No. 5,614,395).

Although most of the studies on SAR have been conducted in dicotyledonous plants, SAR has been demonstrated in monocotyledonous plants as well. For example, SAR has been demonstrated in rice, where an inducing infection by P.s. pv syringae led to systemic protection against Pyricularia oryzae (Smith and Metraux, 1991), the causative agent of leaf blast, and in barley and wheat, where a prior infection by Erysiphe graminis led to enhanced protection against E. graminis, the causative agent of powdery mildew (Schweizer et al., 1989; Hwang and Heitefuss, 1992). Chemically induced resistance by INA has been described in barley (Kogel et al., 1994; Wasternack et al., 1994). More recently, BTH has been shown to induce acquired resistance in wheat against E. graminis, Puccinia recondita, and Septoria spp., and to induce the accumulation of transcripts from a number of novel plant genes that are also shown to be induced during pathogen infection (Görlach et al., 1996).

A number of Arabidopsis mutants have been isolated that have modified SAR signal transduction (Delaney, 1997) The first of these mutants are the so-called lsd (lesions simulating disease) mutants and acd2 (accelerated cell death) (Dietrich et al., 1994; Greenberg et al., 1994). These mutants all have some degree of spontaneous necrotic lesion formation on their leaves, elevated levels of SA, mRNA accumulation for the SAR genes, and significantly enhanced disease resistance. At least seven different lsd mutants have been isolated and characterized (Dietrich et al., 1994; Weymann et al., 1995). Another interesting class of mutants are cim (constitutive immunity) mutants (Lawton et al., 1993). See also, U.S. Pat. No. 5,792,904 and International PCT Application WO 94/16077. Like lsd mutants and acd2, cim mutants have elevated SA and SAR gene expression and resistance, but in contrast to lsd or acd2, do not display detectable lesions on their leaves. cpr1 (constitutive expresser of PR genes) may be a type of cim mutant; however, because the presence of microscopic lesions on the leaves of cpr1 has not been ruled out, cpr1 might be a type of lsd mutant (Bowling et al., 1994).

Mutants have also been isolated that are blocked in SAR signaling. ndr1 (non-race-specific disease resistance) is a mutant that allows growth of both Pseudomonas syringae containing various avirulence genes and also normally avirulent isolates of Peronospora parasitica (Century et al., 1995). Apparently this mutant is blocked early in SAR signaling. npr1 (nonexpresser of PR genes) is a mutant that cannot induce expression of the SAR signaling pathway following INA treatment (Cao et al., 1994). eds (enhanced disease susceptibility) mutants have been isolated based on their ability to support bacterial infection following inoculation of a low bacterial concentration (Glazebrook et al., 1996; Parker et al., 1996). Certain eds mutants are phenotypically very similar to npr1, and, recently, eds5 and eds53 have been shown to be allelic to npr1 (Glazebrook et al., 1996). nim1 (noninducible immunity) is a mutant that supports P. parasitica (i.e., causal agent of downy mildew disease) growth following INA treatment (Delaney et al., 1995; U.S. Pat. No. 5,792,904). Although nim1 can accumulate SA following pathogen infection, it cannot induce SAR gene expression or disease resistance, suggesting that the mutation blocks the pathway downstream of SA. nim1 is also impaired in its ability to respond to INA or BTH, suggesting that the block exists downstream of the action of these chemicals (Delaney et al., 1995; Lawton et al, 1996).

Allelic Arabidopsis genes have been isolated and characterized, mutants of which are responsible for the nim1 and npr1 phenotypes, respectively (Ryals et al., 1997; Cao et al., 1997). The wild-type NIM1 gene product is involved in the signal transduction cascade leading to both SAR and gene-or-gene disease resistance in Arabidopsis (Ryals et al., 1997). Ryals et al., 1997 also report the isolation of five additional alleles of nim1 that show a range of phenotypes from weakly impaired in chemically induced PR-1 gene expression and fungal resistance to very strongly blocked. Transformation of the wild-type NPR1 gene into npr1 mutants not only complemented the mutations, restoring the responsiveness of SAR induction with respect to PR-gene expression and disease resistance, but also rendered the transgenic plants more resistant to infection by P. syringae in the absence of SAR induction (Cao et al., 1997). WO 98/06748 describes the isolation of NPR1 from Arabidopsis and a homologue from Nicotiana glutinosa. See also, WO 97/49822, WO 98/26082, and WO 98/29537. Furthermore, U.S. patent application Ser. No. 09/265,149 of Salmeron et al. describes the isolation of Nicotiana tabacum (tobacco), Lycopersicon esculentum (tomato), Brassica napus (oilseed rape), and Arabidopsis thaliana homologues of the NIM1 gene. Therefore, while NIM1 homologues have been isolated from a number of dicotyledonous plant species, NIM1 homologues have heretofore not been isolated from any monocotyledonous plant species.

Despite much research and the use of sophisticated and intensive crop protection measures, including genetic transformation of plants, losses due to disease remain in the billions of dollars annually. Therefore, there is a continuing need to develop new crop protection measures based on the ever-increasing understanding of the genetic basis for disease resistance in plants. In particular, there is a need for the identification, isolation, and characterization of NIM1 homologues from additional species of plants, particularly monocotyledonous plants.

The present invention addresses the aforementioned needs by providing homologues of the Arabidopsis NIM1 gene from monocotyledonous plant species. In particular, the present invention concerns the isolation of Triticum aestivum (wheat) and Oryza sativa (rice) homologues of the NIM1 gene, which encode proteins believed to be involved in the signal transduction cascade responsive to biological and chemical inducers that lead to systemic acquired resistance in plants.

Hence, the present invention is directed to an isolated nucleic acid molecule comprising a nucleotide sequence from a monocotyledonous plant that is a homologue of the NIM1 gene.

In one particular embodiment, the present invention is directed to an isolated nucleic acid molecule comprising a nucleotide sequence that encodes SEQ ID NO:2, 8, 10, 12, 14, 16, 18, or 20.

In another embodiment, the present invention is directed to an isolated nucleic acid molecule comprising SEQ ID NO:1, 7, 9, 11, 13, 15, 17, or 19.

In a further embodiment, the present invention is directed to an isolated nucleic acid molecule comprising a nucleotide sequence that comprises an at least 20, 25, 30, 35, 40, 45, or 50 (preferably 20) consecutive base pair portion identical in sequence to an at least 20, 25, 30, 35, 40, 45, or 50 (preferably 20) consecutive base pair portion of SEQ ID NO:1, 7, 9, 11, 13, 15,17, or 19.

In yet another embodiment, the present invention is directed to an isolated nucleic acid molecule comprising a nucleotide sequence that can be amplified from a monocotyledonous plant DNA library using the polymerase chain reaction with the pair of primers set forth as SEQ ID NO:3 and 4 or SEQ ID NO:5 and 6.

In still another embodiment, the present invention is directed to an isolated nucleic acid molecule comprising a nucleotide sequence that can be amplified from a Orzya sativa DNA library using the polymerase chain reaction with the pair of primers set forth as SEQ ID NO:3 and 4 or SEQ ID NO:5 and 6.

In yet another embodiment, the present invention is directed to an isolated nucleic acid molecule comprising a nucleotide sequence that can be amplified from a Triticum aestivum DNA library using the polymerase chain reaction with the pair of primers set forth as SEQ ID NO:3 and 4 or SEQ ID NO:5 and 6.

In a further embodiment, the present invention is directed to an isolated nucleic acid molecule comprising a nucleotide sequence that can be amplified from a monocotyledonous plant DNA library using the polymerase chain reaction with a pair of primers comprising the first 20 nucleotides and the reverse complement of the last 20 nucleotides of the coding sequence (CDS) of SEQ ID NO:1, 7, 9, 11, 13, 15, 17, or 19.

In a further embodiment, the present invention is directed to an isolated nucleic acid molecule comprising a nucleotide sequence from a monocotyledonous plant that hybridizes to the complement of SEQ ID NO:1, 7, 9, 11, 13, 15, 17, or 19 under stringent hybridization and wash conditions.

The present invention also encompasses a chimeric gene comprising a promoter active in plants operatively linked to a NIM1 homologue coding sequence of the present invention, a recombinant vector comprising such a chimeric gene, wherein the vector is capable of being stably transformed into a host, as well as a host stably transformed with such a vector. Preferably, the host is a plant such as one of the following agronomically important crops: rice, wheat, barley, rye, canola, sugarcane, corn, potato, carrot, sweet potato, sugar beet, bean, pea, chicory, lettuce, cabbage, cauliflower, broccoli, turnip, radish, spinach, asparagus, onion, garlic, eggplant, pepper, celery, squash, pumpkin, cucumber, apple, pear, quince, melon, plum, cherry, peach, nectarine, apricot, strawberry, grape, raspberry, blackberry, pineapple, avocado, papaya, mango, banana, soybean, tobacco, tomato, sorghum, and sugarcane. More preferably, the host is a monocotyledonous plant. The present invention also encompasses seed from a plant of the invention.

Further, the present invention is directed to a method of increasing SAR gene expression in a plant by expressing in the plant a chimeric gene that itself comprises a promoter active in plants operatively linked to a NIM1 homologue coding sequence of the present invention, wherein the encoded protein is expressed in the transformed plant at higher levels than in a wild type plant. Preferably, the host is a monocotyledonous plant.

In addition, the present invention is directed to a method of enhancing disease resistance in a plant by expressing in the plant a chimeric gene that itself comprises a promoter active in plants operatively linked to a NIM1 homologue coding sequence of the present invention, wherein the encoded protein is expressed in the transformed plant at higher levels than in a wild type plant. Preferably, the host is a monocotyledonous plant.

Further, the present invention is directed to a PCR primer that is SEQ ID NO:3 or 4.

The present invention also encompasses a method for isolating a NIM1 homologue involved in the signal transduction cascade leading to systemic acquired resistance in plants comprising amplifying a DNA molecule from a monocotyledonous plant DNA library using the polymerase chain reaction with a pair of primers corresponding to the first 20 nucleotides and the reverse complement of the last 20 nucleotides of the coding sequence (CDS) of SEQ ID NO:1, 7, 9, 11, 13, 15, 17, or 19 or with the pair of primers set forth as SEQ ID NO:3 and 4 or SEQ ID NO:5 and 6. In a preferred embodiment, the monocotyledonous plant DNA library is a Oryza sativa (rice) or Triticum aestivum (wheat) DNA library.
SEQ ID NO:1—Genomic DNA sequence of a NIM1 homologue (pHW01) from wheat.
SEQ ID NO:2—Protein sequence of the wheat NIM1 homologue encoded by SEQ ID NO:1.
SEQ ID NO:3—Oligonucleotide primer KL1.
SEQ ID NO:4—Oligonucleotide primer KL2.
SEQ ID NO:5—PCR primer NIM 2B.
SEQ ID NO:6—PCR primer NIM 2D.
SEQ ID NO:7—498 bp NIM-like DNA fragment amplified from Oryza sativa (Rice A), which is a consensus of 13 sequences and has 59% sequence identity to the Arabidopsis thaliana NIM1 gene sequence.
SEQ ID NO:8—Protein sequence encoded by SEQ ID NO:7.
SEQ ID NO:9—498 bp NIM-like DNA fragment amplified from Oryza sativa (Rice B), which has 62% sequence identity to the Arabidopsis thaliana NIM1 gene sequence.
SEQ ID NO:10—Protein sequence encoded by SEQ ID NO:9.
SEQ ID NO:11—498 bp NIM-like DNA fragment amplified from Triticum aestivum (Wheat), which is a consensus of 3 sequences and has 55% sequence identity to the Arabidopsis thaliana NIM1 gene sequence.
SEQ ID NO:12—Protein sequence encoded by SEQ ID NO:11.
SEQ ID NO:13—Full length cDNA sequence of a NIM1 homologue from Oryza sativa (Rice A), which corresponds to the PCR fragment of SEQ ID NO:7.
SEQ ID NO:14—Protein sequence of the rice NIM1 homologue encoded by SEQ ID NO:13.
SEQ ID NO:15—Partial cDNA sequence of a NIM1 homologue from Oryza sativa (Rice B), which corresponds to the PCR fragment of SEQ ID NO:9.
SEQ ID NO:16—Protein sequence of the rice NIM1 homologue encoded by SEQ ID NO:15.
SEQ ID NO:17—Full length cDNA sequence of a NIM1 homologue from Triticum aestivum (Wheat), which corresponds to the PCR fragment of SEQ ID NO:11.
SEQ ID NO:18—Protein sequence of the wheat NIM1 homologue encoded by SEQ ID NO:17.
SEQ ID NO:19—Full length cDNA sequence corresponding to the Triticum aestivum (wheat) NIM-like genomic sequence pHW01 (SEQ ID NO:1).
SEQ ID NO:20—Protein sequence encoded by SEQ ID NO:19.

In describing the present invention, the following terms may be employed, and are intended to be defined as indicated below.

Associated With/Operatively Linked: Refers to two DNA sequences that are related physically or functionally. For example, a promoter or regulatory DNA sequence is said to be “associated with” a DNA sequence that codes for an RNA or a protein if the two sequences are operatively linked, or situated such that the regulator DNA sequence will affect the expression level of the coding or structural DNA sequence.

Chimeric Gene: A recombinant DNA sequence in which a promoter or regulatory DNA sequence is operatively linked to, or associated with, a DNA sequence that codes for an mRNA or which is expressed as a protein, such that the regulator DNA sequence is able to regulate transcription or expression of the associated DNA sequence. The regulator DNA sequence of the chimeric gene is not normally operatively linked to the associated DNA sequence as found in nature.

Coding Sequence: a nucleic acid sequence that is transcribed into RNA such as mRNA, rRNA, tRNA, snRNA, sense RNA or antisense RNA. Preferably the RNA is then translated in an organism to produce a protein.

Complementary: refers to two nucleotide sequences that comprise antiparallel nucleotide sequences capable of pairing with one another upon formation of hydrogen bonds between the complementary base residues in the antiparallel nucleotide sequences.

Expression: refers to the transcription and/or translation of an endogenous gene or a transgene in plants. In the case of antisense constructs, for example, expression may refer to the transcription of the antisense DNA only.

Expression Cassette: A nucleic acid sequence capable of directing expression of a particular nucleotide sequence in an appropriate host cell, comprising a promoter operatively linked to the nucleotide sequence of interest which is operatively linked to termination signals. It also typically comprises sequences required for proper translation of the nucleotide sequence. The expression cassette comprising the nucleotide sequence of interest may be chimeric, meaning that at least one of its components is heterologous with respect to at least one of its other components. The expression cassette may also be one which is naturally occurring but has been obtained in a recombinant form useful for heterologous expression. Typically, however, the expression cassette is heterologous with respect to the host, i.e., the particular nucleic acid sequence of the expression cassette does not occur naturally in the host cell and must have been introduced into the host cell or an ancestor of the host cell by a transformation event. The expression of the nucleotide sequence in the expression cassette may be under the control of a constitutive promoter or of an inducible promoter which initiates transcription only when the host cell is exposed to some particular external stimulus. In the case of a multicellular organism, such as a plant, the promoter can also be specific to a particular tissue, or organ, or stage of development.

Gene: A defined region that is located within a genome and that, besides the aforementioned coding nucleic acid sequence, comprises other, primarily regulatory, nucleic acid sequences responsible for the control of expression, i.e., transcription and translation of the coding portion. A gene may also comprise other 5′ and 3′ untranslated sequences and termination sequences. Further elements that may be present are, for example, introns.

Heterologous DNA Sequence: The terms “heterologous DNA sequence”, “exogenous DNA segment” or “heterologous nucleic acid,” as used herein, each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also includes non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides.

Homologous DNA Sequence: A DNA sequence naturally associated with a host cell into which it is introduced.

Isocoding: A nucleic acid sequence is isocoding with a reference nucleic acid sequence when the nucleic acid sequence encodes a polypeptide having the same amino acid sequence as the polypeptide encoded by the reference nucleic acid sequence.

Isolated: In the context of the present invention, an isolated nucleic acid molecule or an isolated enzyme is a nucleic acid molecule or enzyme that, by the hand of man, exists apart from its native environment and is therefore not a product of nature. An isolated nucleic acid molecule or enzyme may exist in a purified form or may exist in a non-native environment such as, for example, a recombinant host cell.

Minimal Promoter: a promoter element, particularly a TATA element, that is inactive or has greatly reduced promoter activity in the absence of upstream activation. In the presence of a suitable transcription factor, a minimal promoter functions to permit transcription.

Native: refers to a gene that is present in the genome of an untransformed cell.

Naturally occurring: the term “naturally occurring” is used to describe an object that can be found in nature as distinct from being artificially produced by man. For example, a protein or nucleotide sequence present in an organism (including a virus), which can be isolated from a source in nature and which has not been intentionally modified by man in the laboratory, is naturally occurring.

NIM1: Gene described in Ryals et al., 1997, which is involved in the SAR signal transduction cascade.

NIM1: Protein encoded by the NIM1 gene

Nucleic acid: the term “nucleic acid” refers to deoxyribonucleotides or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g. degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19: 5081 (1991); Ohtsuka et al., J. Biol. Chem. 260: 2605-2608 (1985); Rossolini et al., Mol. Cell. Probes 8: 91-98 (1994)). The terms “nucleic acid” or “nucleic acid sequence” may also be used interchangeably with gene, cDNA, and mRNA encoded by a gene. In the context of the present invention, the nucleic acid molecule is preferably a segment of DNA. Nucleotides are indicated by their bases by the following standard abbreviations: adenine (A), cytosine (C), thymine M, and guanine (G).

ORF: Open Reading Frame.

Plant: Any whole plant.

Plant Cell: Structural and physiological unit of a plant, comprising a protoplast and a cell wall. The plant cell may be in form of an isolated single cell or a cultured cell, or as a part of higher organized unit such as, for example, a plant tissue, a plant organ, or a whole plant.

Plant Cell Culture: Cultures of plant units such as, for example, protoplasts, cell culture cells, cells in plant tissues, pollen, pollen tubes, ovules, embryo sacs, zygotes and embryos at various stages of development.

Plant Material: Refers to leaves, stems, roots, flowers or flower parts, fruits, pollen, egg cells, zygotes, seeds, cuttings, cell or tissue cultures, or any other part or product of a plant.

Plant Organ: A distinct and visibly structured and differentiated part of a plant such as a root, stem, leaf, flower bud, or embryo.

Plant tissue: A group of plant cells organized into a structural and functional unit. Any tissue of a plant in planta or in culture is included. This term includes, but is not limited to, whole plants, plant organs, plant seeds, tissue culture and any groups of plant cells organized into structural and/or functional units. The use of this term in conjunction with, or in the absence of, any specific type of plant tissue as listed above or otherwise embraced by this definition is not intended to be exclusive of any other type of plant tissue.

Promoter: An untranslated DNA sequence upstream of the coding region that contains the binding site for RNA polymerase II and initiates transcription of the DNA. The promoter region may also include other elements that act as regulators of gene expression.

Protoplast: An isolated plant cell without a cell wall or with only parts of the cell wall.

Purified: the term “purified,” when applied to a nucleic acid or protein, denotes that the nucleic acid or protein is essentially free of other cellular components with which it is associated in the natural state. It is preferably in a homogeneous state although it can be in either a dry or aqueous solution. Purity and homogeneity are typically determined using analytical chemistry techniques such as polyacrylamide gel electrophoresis or high performance liquid chromatography. A protein which is the predominant species present in a preparation is substantially purified. The term “purified” denotes that a nucleic acid or protein gives rise to essentially one band in an electrophoretic gel. Particularly, it means that the nucleic acid or protein is at least about 50% pure, more preferably at least about 85% pure, and most preferably at least about 99% pure.

Recombinant DNA molecule: a combination of DNA molecules that are joined together using recombinant DNA technology Regulatory Elements: Sequences involved in controlling the expression of a nucleotide sequence. Regulatory elements comprise a promoter operably linked to the nucleotide sequence of interest and termination signals. They also typically encompass sequences required for proper translation of the nucleotide sequence.

Selectable marker gene: a gene whose expression in a plant cell gives the cell a selective advantage. The selective advantage possessed by the cells transformed with the selectable marker gene may be due to their ability to grow in the presence of a negative selective agent, such as an antibiotic or a herbicide, compared to the growth of non-transformed cells. The selective advantage possessed by the transformed cells, compared to non-transformed cells, may also be due to their enhanced or novel capacity to utilize an added compound as a nutrient, growth factor or energy source. Selectable marker gene also refers to a gene or a combination of genes whose expression in a plant cell gives the cell both, a negative and a positive selective advantage.

Significant Increase: an increase in enzymatic activity that is larger than the margin of error inherent in the measurement technique, preferably an increase by about 2-fold or greater of the activity of the wild-type enzyme in the presence of the inhibitor, more preferably an increase by about 5-fold or greater, and most preferably an increase by about 10-fold or greater.

The terms “identical” or percent “identity” in the context of two or more nucleic acid or protein sequences, refer to two or more sequences or subsequences that are the same or have a specified percentage of amino acid residues or nucleotides that are the same, when compared and aligned for maximum correspondence, as measured using one of the following sequence comparison algorithms or by visual inspection.

Substantially identical: the phrase “substantially identical,” in the context of two nucleic acid or protein sequences, refers to two or more sequences or subsequences that have at least 60%, preferably 80%, more preferably 90-95%, and most preferably at least 99% nucleotide or amino acid residue identity, when compared and aligned for maximum correspondence, as measured using one of the following sequence comparison algorithms or by visual inspection. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions. Furthermore, substantially identical nucleic acid or protein sequences perform substantially the same function.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2: 482 (1981), by the homology alignment algorithm of Needleman & Wunsch, J. Mol. Biol. 48: 443 (1970), by the search for similarity method of Pearson & Lipman, Proc. Nat'l. Acad. Sci. USA 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Ausubel et al., infra).

One example of an algorithm that is suitable for determining percent sequence identity and sequence similarity is the BLAST algorithm, which is described in Altschul et al., J. Mol. Biol. 215: 403-410 (1990). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90: 5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

Another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. An extensive guide to the hybridization of nucleic acids is found in Tijssen (1993) Laboratory Techniques in Biochemistry and Molecular Biology-Hybridization with Nucleic Acid Probes part I chapter 2 “Overview of principles of hybridization and the strategy of nucleic acid probe assays” Elsevier, N.Y. Generally, highly stringent hybridization and wash conditions are selected to be about 5° C. lower than the thermal melting point (Tₘ) for the specific sequence at a defined ionic strength and pH. Typically, under “stringent conditions” a probe will hybridize to its target subsequence, but to no other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0M Na ion, typically about 0.01 to 1.0 M Na ion concentration (or other salts) at pH 7.0 to 8.3, and the temperature is typically at least about 30-C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

The following are examples of sets of hybridization/wash conditions that may be used to clone homologous nucleotide sequences that are substantially identical to reference nucleotide sequences of the present invention: a reference nucleotide sequence preferably hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% sodium dodecyl sulfate (SDS), 0.5 M NaPO₄, 1 mM EDTA at 50° C. with washing in 1×SSC, 0.1% SDS at 50° C., more desirably still in 7% sodium dodecyl sulfate (SDS), 0.5 M NaPO₄, 1 mM EDTA at 50° C. with washing in 0.5×SSC, 0.1% SDS at 50° C., preferably in 7% sodium dodecyl sulfate (SDS), 0.5 M NaPO₄, 1 mM EDTA at 50° C. with washing in 0.1×SSC, 0.1% SDS at 50° C., more preferably in 7% sodium dodecyl sulfate (SDS), 0.5 M NaPO₄, 1 mM EDTA at 50° C. with washing in 0.1×SSC, 0.1% SDS at 65° C.

A further indication that two nucleic acid sequences or proteins are substantially identical is that the protein encoded by the first nucleic acid is immunologically cross reactive with, or specifically binds to, the protein encoded by the second nucleic acid. Thus, a protein is typically substantially identical to a second protein, for example, where the two proteins differ only by conservative substitutions.

The phrase “specifically (or selectively) binds to an antibody,” or “specifically (or selectively) immunoreactive with,” when referring to a protein or peptide, refers to a binding reaction which is determinative of the presence of the protein in the presence of a heterogeneous population of proteins and other biologics. Thus, under designated immunoassay conditions, the specified antibodies bind to a particular protein and do not bind in a significant amount to other proteins present in the sample. Specific binding to an antibody under such conditions may require an antibody that is selected for its specificity for a particular protein. For example, antibodies raised to the protein with the amino acid sequence encoded by any of the nucleic acid sequences of the invention can be selected to obtain antibodies specifically immunoreactive with that protein and not with other proteins except for polymorphic variants. A variety of immunoassay formats may be used to select antibodies specifically immunoreactive with a particular protein. For example, solid-phase ELISA immunoassays, Western blots, or immunohistochemistry are routinely used to select monoclonal antibodies specifically immunoreactive with a protein. See Harlow and Lane (1988) Antibodies, A Laboratory Manual, Cold Spring Harbor Publications, New York “Harlow and Lane”), for a description of immunoassay formats and conditions that can be used to determine specific immunoreactivity. Typically a specific or selective reaction will be at least twice background signal or noise and more typically more than 10 to 100 times background.

“Conservatively modified variations” of a particular nucleic acid sequence refers to those nucleic acid sequences that encode identical or essentially identical amino acid sequences, or where the nucleic acid sequence does not encode an amino acid sequence, to essentially identical sequences. Because of the degeneracy of the genetic code, a large number of functionally identical nucleic acids encode any given polypeptide. For instance the codons CGT, CGC, CGA, CGG, AGA, and AGG all encode the amino acid arginine. Thus, at every position where an arginine is specified by a codon, the codon can be altered to any of the corresponding codons described without altering the encoded protein. Such nucleic acid variations are “silent variations” which are one species of “conservatively modified variations.” Every nucleic acid sequence described herein which encodes a protein also describes every possible silent variation, except where otherwise noted. One of skill will recognize that each codon in a nucleic acid (except ATG, which is ordinarily the only codon for methionine) can be modified to yield a functionally identical molecule by standard techniques. Accordingly, each “silent variation” of a nucleic acid which encodes a protein is implicit in each described sequence.

Furthermore, one of skill will recognize that individual substitutions deletions or additions that alter, add or delete a single amino acid or a small percentage of amino acids (typically less than 5%, more typically less than 1%) in an encoded sequence are “conservatively modified variations,” where the alterations result in the substitution of an amino acid with a chemically similar amino acid. Conservative substitution tables providing functionally similar amino acids are well known in the art. The following five groups each contain amino acids that are conservative substitutions for one another: Aliphatic: Glycine (G), Alanine (A), Valine (V), Leucine (L), Isoleucine (I); Aromatic: Phenylalanine (F), Tyrosine (Y), Tryptophan (W); Sulfur-containing: Methionine (M), Cysteine (C); Basic: Arginine (R), Lysine (K), Histidine (H); Acidic: Aspartic acid (D), Glutamic acid (E), Asparagine (N), Glutamine (Q). See also, Creighton (1984) Proteins, W.H. Freeman and Company. In addition, individual substitutions, deletions or additions which alter, add or delete a single amino acid or a small percentage of amino acids in an encoded sequence are also “conservatively modified variations.”

A “subsequence” refers to a sequence of nucleic acids or amino acids that comprise a part of a longer sequence of nucleic acids or amino acids (e.g., protein) respectively.

Nucleic acids are “elongated” when additional nucleotides (or other analogous molecules) are incorporated into the nucleic acid. Most commonly, this is performed with a polymerase (e.g., a DNA polymerase), e.g., a polymerase which adds sequences at the 3′ terminus of the nucleic acid.

Two nucleic acids are “recombined” when sequences from each of the two nucleic acids are combined in a progeny nucleic acid. Two sequences are “directly” recombined when both of the nucleic acids are substrates for recombination. Two sequences are “indirectly recombined” when the sequences are recombined using an intermediate such as a cross-over oligonucleotide. For indirect recombination, no more than one of the sequences is an actual substrate for recombination, and in some cases, neither sequence is a substrate for recombination.

A “specific binding affinity” between two molecules, for example, a ligand and a receptor, means a preferential binding of one molecule for another in a mixture of molecules. The binding of the molecules can be considered specific if the binding affinity is about 1×10⁴ M⁻¹ to about 1×10⁶ M⁻¹ or greater.

Transformation: a process for introducing heterologous DNA into a host cell or organism.

“Transformed,” “transgenic,” and “recombinant” refer to a host organism such as a bacterium or a plant into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed,” “non-transgenic,” or “non-recombinant” host refers to a wild-type organism, e.g., a bacterium or plant, which does not contain the heterologous nucleic acid molecule.

The following material has been deposited with the Agricultural Research Service, Patent Culture Collection (NRRL), 1815 North University Street, Peoria, Ill. 61604, USA, under the terms of the Budapest Treaty on the International Recognition of the Deposit of Microorganisms for the Purposes of Patent Procedure. All restrictions on the availability of the deposited material will be irrevocably removed upon the granting of a patent.

| Clone | Accession Number | Date of Deposit |
|---|---|---|
| pHW01 | NRRL B-30152 | Jul. 1, 1999 |

The present invention concerns monocotyledonous NIM1 homologues, such as those isolated from Triticum aestivum (wheat) and Oryza sativa (rice). As described more fully below in the Examples, monocotyledonous NIM1 homologues according to the invention may be isolated from cDNA and/or genomic DNA libraries by probing with fragments of the tobacco NIM1 cDNA described in WO 00/53762, the disclosure of which is hereby incorporated by reference in its entirety.

In addition, NIM1 homologues according to the invention can be isolated from cDNA and/or genomic DNA libraries from monocotyledonous plants by PCR amplification using primers constructed based on the NIM1 sequences from Arabidopsis thaliana, Nicotiana tabacum, and Lycopersicon esculentum, as well as the NML sequences from Arabidopsis thaliana (see, Example 5: “Design of Degenerate Primers” in WO 00/53762).

Furthermore, monocotyledonous NIM1 homologues according to the invention can be isolated by PCR using the wheat and rice sequences set forth in the attached sequence listing as the basis for constructing PCR primers. For example, the first and last 20-25 consecutive nucleotides of SEQ ID NO:19 (e.g., nucleotides 1-20 and 1649-1668 of SEQ ID NO:19) can be used as the basis for constructing PCR primers to amplify the cDNA sequence (SEQ ID NO:19) directly from a cDNA library from the source plant (wheat). Other DNA sequences of the invention can likewise be amplified by PCR from cDNA or genomic DNA libraries of monocotyledonous plants using the ends of the DNA sequences set forth in the sequence listing as the basis for PCR primers.

Monocotyledonous NIM1 homologues, such as the wheat and rice NIM1 homologues described herein, are predicted to encode proteins involved in the signal transduction cascade responsive to biological and chemical inducers, which leads to systemic acquired resistance in plants. The present invention also concerns the transgenic expression of a monocotyledonous NIM1 homologue in plants to increase SAR gene expression and enhance disease resistance.

The transgenic expression of a monocotyledonous NIM1 homologue of the invention in plants is predicted to result in immunity to a wide array of plant pathogens, which include, but are not limited to viruses or viroids, e.g. tobacco or cucumber mosaic virus, ringspot virus or necrosis virus, pelargonium leaf curl virus, red clover mottle virus, tomato bushy stunt virus, and like viruses; fungi, e.g. oomycetes such as Phythophthora parasitica and Peronospora tabacina; bacteria, e.g. Pseudomonas syringae and Pseudomonas tabaci; insects such as aphids, e.g. Myzus persicae; and lepidoptera, e.g., Heliothus spp.; and nematodes, e.g., Meloidogyne incognita. The vectors and methods of the invention are useful against a number of disease organisms of maize including but not limited to downy mildews such as Scleropthora macrospora, Sclerophthora rayissiae, Sclerospora graminicola, Peronosclerospora sorghi, Peronoscierospora philippinensis, Peronosclerospora sacchari and Peronosclerospora maydis; rusts such as Puccinia sorphi, Puccinia polysora and Physopella zeae; other fungi such as Cercospora zeae-maydis, Colletotrichum graminicola, Fusarium monoliforme, Gibberella zeae, Exserohilum turcicum, Kabatiellu zeae, Erysiphe graminis, Septoria and Bipolaris maydis; and bacteria such as Erwinia stewartii.

The methods of the present invention can be utilized to confer disease resistance to a wide variety of plants, including gymnosperms, monocots, and dicots. Although disease resistance can be conferred upon any plants falling within these broad classes, it is particularly useful in agronomically important crop plants, such as rice, wheat, barley, rye, rape, corn, potato, carrot, sweet potato, sugar beet, bean, pea, chicory, lettuce, cabbage, cauliflower, broccoli, turnip, radish, spinach, asparagus, onion, garlic, eggplant, pepper, celery, carrot, squash, pumpkin, zucchini, cucumber, apple, pear, quince, melon, plum, cherry, peach, nectarine, apricot, strawberry, grape, raspberry, blackberry, pineapple, avocado, papaya, mango, banana, soybean, tobacco, tomato, sorghum and sugarcane.

A monocotyledonous NIM1 homologue coding sequence of the present invention may be inserted into an expression cassette designed for plants to construct a chimeric gene according to the invention using standard genetic engineering techniques. The choice of specific regulatory sequences such as promoter, signal sequence, 5′ and 3′ untranslated sequences, and enhancer appropriate for the achieving the desired pattern and level of expression in the chosen plant host is within the level of skill of the routineer in the art. The resultant molecule, containing the individual elements linked in proper reading frame, may be inserted into a vector capable of being transformed into a host plant cell.

Examples of promoters capable of functioning in plants or plant cells (i.e., those capable of driving expression of associated coding sequences such as those coding for NIM1 homologues in plant cells) include the Arabidopsis and maize ubiquitin promoters; cauliflower mosaic virus (CaMV) 19S or 35S promoters and CaMV double promoters; rice actin promoters; PR-1 promoters from tobacco, Arabidopsis, or maize; nopaline synthase promoters; small subunit of ribulose bisphosphate carboxylase (ssuRUBISCO) promoters, and the like. Especially preferred is the Arabidopsis ubiquitin promoter. The promoters themselves may be modified to manipulate promoter strength to increase expression of the associated coding sequence in accordance with art-recognized procedures. Preferred promoters for use with the present invention are those that confer high level constitutive expression.

Signal or transit peptides may be fused to the monocotyledonous NIM1 homologue coding sequence in the chimeric DNA constructs of the invention to direct transport of the expressed protein to the desired site of action. Examples of signal peptides include those natively linked to the plant pathogenesis-related proteins, e.g. PR-1, PR-2, and the like. See, e.g., Payne et al., 1988. Examples of transit peptides include the chloroplast transit peptides such as those described in Von Heijne et al. (1991), Mazur et al. (1987), and Vorst et al. (1988); and mitochondrial transit peptides such as those described in Boutry et al. (1987). Also included are sequences that result in localization of the encoded protein to various cellular compartments such as the vacuole. See, for example, Neuhaus et al. (1991) and Chrispeels (1991).

The chimeric DNA construct(s) of the invention may contain multiple copies of a promoter or multiple copies of a monocotyledonous NIM1 homologue coding sequence of the present invention. In addition, the construct(s) may include coding sequences for markers and coding sequences for other peptides such as signal or transit peptides, each in proper reading frame with the other functional elements in the DNA molecule. The preparation of such constructs are within the ordinary level of skill in the art.

Useful markers include peptides providing herbicide, antibiotic or drug resistance, such as, for example, resistance to protoporphyrinogen oxidase inhibitors, hygromycin, kanamycin, G418, gentamycin, lincomycin, methotrexate, glyphosate, phosphinothricin, or the like. These markers can be used to select cells transformed with the chimeric DNA constructs of the invention from untransformed cells. Other useful markers are peptidic enzymes which can be easily detected by a visible reaction, for example a color reaction, for example luciferase, β-glucuronidase, or β-galactosidase.

Chimeric genes designed for plant expression such as those described herein can be introduced into the plant cell in a number of art-recognized ways. Those skilled in the art will appreciate that the choice of method might depend on the type of plant (i.e. monocot or dicot) and/or organelle (i.e. nucleus, chloroplast, mitochondria) targeted for transformation. Suitable methods of transforming plant cells include microinjection (Crossway et al., 1986), electroporation (Riggs et al., 1986), Agrobacterium mediated transformation (Hinchee et al., 1988; Ishida et al., 1996), direct gene transfer (Paszkowski et al., 1984; Hayashimoto et al., 1990), and ballistic particle acceleration using devices available from Agracetus, Inc., Madison, Wis. and Dupont, Inc., Wilmington, Del. (see, for example, U.S. Pat. 4,945,050; and McCabe et al., 1988). See also, Weissinger et al. (1988); Sanford et al. (1987) (onion); Christou et al. (1988) (soybean); McCabe et al. (1988) (soybean); Datta et al. (1990) (rice); Klein et al. (1988) (maize); Klein et al. (1988) (maize); Klein et al. (1988) (maize); Fromm et al. (1990); and Gordon-Kamm et al. (1990) (maize); Svab et al. (1990) (tobacco chloroplasts); Gordon-Kamm et al. (1 993) (maize); Shimamoto et al. (1 989) (rice); Christou et al. (1991) (rice); Datta et al. (1990) (rice); European Patent Application EP 0 332 581 (orchardgrass and other Pooideae); Vasil et al. (1993) (wheat); Weeks et al. (1993) (wheat); Wan et al. (1994) (barley); Jahne et al. (1994) (barley); Umbeck et al. (1987) (cotton); Casas et al. (1993) (sorghum); Somers et al. (1992) (oats); Torbert et al. (1995) (oats); Weeks et al.,(1 993) (wheat); WO 94/13822 (wheat); and Nehra et al. (1994) (wheat). A particularly preferred set of embodiments for the introduction of recombinant DNA molecules into maize by microprojectile bombardment can be found in Koziel et al. (1993); Hill et al. (1995) and Koziel et al. (1996). An additional preferred embodiment is the protoplast transformation method for maize as disclosed in EP 0 292 435.

Once a chimeric gene comprising a monocotyledonous NIM1 homologue coding sequence has been transformed into a particular plant species, it may be propagated in that species or moved into other varieties of the same species, particularly including commercial varieties, using traditional breeding techniques. Particularly preferred plants of the invention include the agronomically important crops listed above. The genetic properties engineered into the transgenic seeds and plants described above are passed on by sexual reproduction and can thus be maintained and propagated in progeny plants.

### EXAMPLES

The invention is illustrated in further detail by the following detailed procedures, preparations, and examples. The examples are for illustration only, and are not to be construed as limiting the scope of the present invention. Standard recombinant DNA and molecular cloning techniques used here are well known in the art and are described by Sambrook, et al., 1989; by T. J. Silhavy, M. L. Berman, and L. W. Enquist, 1984; and by Ausubel, F. M. et al., 1987.

I. Isolation of Homologues of the Arabidopsis NIM1 Gene from Monocotyledonous Plants

### Example 1

### Isolation of a NIM1 Homologue from Triticum aestivum (Wheat)

A custom genomic DNA library from Triticum aestivum (cv UC703) is constructed in EMBL3 SP6/T7 vectors (Clontech). The library (1×10⁶ pfu) is screened following the protocol of Clontech Laboratories. Two different fragments of the tobacco NIM1 cDNA (pNOV1206—SEQ ID NO:1 of WO 00/53762) are used as probes: the 5′-NIM1 fragment (nucleotide seq. 1-790; 0.8 kb AccI/EcoRI fragment isolated from pNOV1206) and the 3′-NIM1 fragment (nucleotide seq. 1176-1770; 0.6 kb KpnI/HindIII fragment isolated from pNOV1206). Plaque lifts, each containing 50,000 clones, a total of 1×10⁶ clones (nitrocellulose membranes, NEN), are made in duplicate from 10 phage plates and each probe is hybridized to 10 membranes. The probe is labeled with P32-dCTP using the method of Prime-ItR II Random Primer Labeling (Stratagene). Hybridization is preferably carried out at 58° C. in hybridization buffer (6×SSPE, 5×Denhards, 0.5% SDS, 100 μg stDNA/ml), and washings are preferably conducted in (I): 2×SSPE, SDS 0.1%, room temperature 10 min, (II): 2×SSPE, SDS 0.1% at 55° C. 15 min, and (III) 1×SSPE, SDS 0.1% at 55° C. 15 min, twice for each washing. A total of nine positive clones are isolated by two additional rounds of plaque purification.

Lambda phage DNA is isolated from K802 lysates according to Zabarovsky and Turina, 1988. Among nine positive candidates, six hybridize to both 3′-NIM1 and 5′- NIM1 probes by Southern blotting of restriction digested lambda DNA. Hybridizing DNA fragments are then cloned into pUC19 vector (NEB).

DNA sequence of clone HW01 is determined by primer walking using 18-mers designed on the ABI 3948 DNA Synthesizer. HW01 template is sequenced with Big Dye Terminator Sequencing Reactions, using 400 ng template per reaction. Cycle conditions are according to the DT 50-30 Program: 95° C.-10 sec. 50° C.-5 sec, 60° C.-4 min for 29 cycles. Following the thermal cycle condition program, the reactions are precipitated with isopropanol. Samples are loaded onto a polyacrylamide gel and analyzed on the ABI 377 Automated Sequencer.

The HW01 template is also subjected to a Primer Island protocol whereby template is prepared on the Qiagen Robot and sequenced in a 96-well Marsh plate block format. Primers used for the plate sequencing are forward and reverse primers from the Primer Island Kit. Sequencing data is analyzed and assembled using Phred/Phrap and Consed Programs.

One of the subcloned DNA sequences from part of lambda clone #8, named pHW01, bears a 4270 bp SacI insert and is identified as a wheat homologue of the Arabidopsis NIM1 gene (Ryals et al., 1997). Translated amino acid sequence of the wheat NIM1 homologue is based on inverted sequence of HW01 (i-HW01) in which the orientation of the NIM1 homologue is the same as the Arabidopsis NIM1 sequence. The wheat NIM1 amino acid sequence has 77/68% amino acid similarity/identity to the tobacco NIM1 homologue shown as SEQ ID NO:1 of WO 00/53762, 78/68% to the tomato NIM1 homologue shown as SEQ ID NO:3 of WO 00/53762, 65/51% to Arabidopsis NIM1 (Ryals et al., 1997), and 69%, 69%, and 59% nucleotide similarity to the tobacco, tomato, and Arabidopsis NIM1 genes, respectively (see, Table 1 and Table 2, below).

TABLE 1
Amino Acid Comparison (Similarity/Identity) of NIM1 Homologues

| | Wheat | Tobacco | Tomato | Arabidopsis |
|---|---|---|---|---|
| Wheat | 100 | | | |
| Tobacco | 77/68 | 100 | | |
| Tomato | 78/68 | 93/90 | 100 | |
| Arabidopsis | 65/51 | 65/54 | 66/55 | 100 |

TABLE 2
Nucleotide Comparison (Identity) of NIM1 Homologues

| | Wheat | Tobacco | Tomato | Arabidopsis |
|---|---|---|---|---|
| Wheat | 100 | | | |
| Tobacco | 69 | 100 | | |
| Tomato | 69 | 90 | 100 | |
| Arabidopsis | 59 | 63 | 62 | 100 |

The genomic sequence of the wheat NIM1 homologue is shown in SEQ ID NO:1 and the encoded protein sequence is shown in SEQ ID NO:2. The wheat NIM1 homologue comprising SEQ ID NO:1 was deposited in E. coli DH5α as pHW01 with the NRRL (Agricultural Research Service, Patent Culture Collection, Northern Regional Research Center, 1815 North University Street, Peoria, Ill. 61604, U.S.A) on Jul. 1, 1999, and assigned accession no. NRRL B-30152.

### Example 2

### PCR Amplification of the Wheat NIM1 Homologue

PCR is used to confirm that the wheat NIM1 homologue originates from the wheat genome. Primers KL1 (19 nt, 5′-CCATTGCTACTCTTGCCTC-3′ (SEQ ID NO:3)) and KL2 (21 nt, 5′-ATCGTTGTCTCCCTTTTAACC-3′ (SEQ ID NO:4)) corresponding to nucleotides 1871-1890 and nucleotides 2360-2340, respectively, from the pHW01 subclone sequence are used to prime PCR reactions using wheat UC703 genomic DNA as template. Cycling conditions are 94° C. for 30 sec, 50° C. for 30 sec, and 72° C. for 30 sec, for a total of 35 cycles. A ˜500 bp band is obtained and cloned. Sequencing of multiple clones with the correct sized insert reveal that three different sequences are amplified from the wheat genome. All three sequences are highly similar to each other, and one of the sequences aligns precisely with the corresponding region of HW01, indicating the HW01 in fact originates from the wheat genome. A wheat NIM1 homologue according to the invention can therefore be isolated by PCR from a wheat genomic library using the above-described PCR primers, KL1 and KL2.

### Example 3

### Isolation of Monocotyledonous NIM1 Homologues by Southern Hybridization

DNA from a monocotyledonous plant is isolated using the miniprep method of Dellaporta et al. (1983). Southern blotting is performed according to the standard protocol (Amersharm). The DNA sequence of the wheat NIM1 homologue corresponding to the NIM1-specific “NIM loop” (nucleotides 2180-3251 of i-HW01, a 1.1 kb NdeI/BgIII fragment isolated from pHW01) is hybridized to genomic DNA of wheat (cv. UC703) and other monocotyledonous crops (e.g., rice, barley and corn). Hybridization is preferably performed at 65° C. in 5×SSPE, 5×Denhards, 0.5% SDS, 100 μg stDNA/ml, and washing is preferably (I): 2×SSPE, SDS 0.1%, room temperature 10 min, (II): 0.2×SSPE, SDS 0.1% at 65° C. 15 min, and (III) 0.1×SSPE, SDS 0.1% at 65° C. 15 min, twice for each washing. The monocotyledonous crops tested show strong hybridization signals to the wheat NIM1 sequence, indicating the presence of NIM1 homologues in these crops. Hybridization signals in wheat genomic DNA indicate that at least four NIM1 homologues are present in the wheat genome.

The PCR product from wheat genomic DNA that is obtained with PCR primers KL1 and KL2 (SEQ ID NO:3 and SEQ ID NO:4, respectively) is used to probe gel blots of wheat RNA. Hybridization with total RNA reveals one faint transcript. However, hybridization with polyA⁺ RNA reveals the presence of two transcripts: a smaller, more abundant mRNA transcript and a larger, less abundant mRNA. The smaller transcript corresponds to the size detected in total RNA. Both transcripts appear to be present in equal abundance in RNA isolated from leaf tissue from young wheat plants that are untreated or BTH-treated for 24 h. The wheat “NIM loop”-described above is also used as a probe.

### Example 4

### Isolation of NIM1 Homologues by PCR from Genomic DNA Libraries of Monocotyledonous Crops

Primers KL1 and KL2 (SEQ ID NO:3 and SEQ ID NO:4, respectively) are used to clone NIM1 homologues from other monocotyledonous crops. Using the same cycling conditions as used for wheat genomic DNA amplification (Example 2), bands of approximately 500 bp in size are amplified from rice, corn, and barley genomic DNA libraries. The PCR products from the rice DNA are cloned and sequenced. All sequenced clones are found to contain the same insert, and the sequence of the insert shows strong similarity to the Arabidopsis NIM1 gene and its crop homologues, indicating that a rice homologue of NIM1 has been cloned.

### Example 5

### Isolation of NIM1 Homologues by PCR from cDNA Libraries of Monocotyledonous Crops

Degenerate PCR primers are designed based on conserved regions discovered by using the GCG Seqweb multiple sequence alignment program (Pretty, Wisconsin Genetics Computer Group) to align the Arabidopsis NIM1 gene (Ryals et al., 1997); the Arabidopsis thaliana NIM-like (NML) genomic sequences AtNMLc5, AtNMLc2, AtNMLc4-1, and AtNMLc4-2; and the NIM1 sequences from Nicotiana tabacum and Lycopersicon esculentum (See WO 00/53762). Based on this alignment, degenerate PCR primers are designed for PCR amplification of NIM1 homologues from other crop species including wheat and rice. Two of the primers designed from these conserved regions are listed below in Table 3. Primers are preferably synthesized by Genosys Biotechnologies, Inc. (The Woodlands, Tex.). Positions of degeneracy are indicated in Table 3 by the notation of more than one base at a single site in the oligonucleotide. “Orientation” designates whether the primer is directed towards the 3′ end (Downstream) or the 5′ end (Upstream) of the cDNA.

TABLE 3
Degenerate Primers

| | | | |
|---|---|---|---|
| Primer | Sequence (5′ to 3′) | SEQ ID NO: | Orientation |

| | | | |
|---|---|---|---|
| NIM 2B | GGCACTGGACTCAGATGATGTTGAACT | SEQ ID NO:5 | Downstream |
| | T T T GT | | |
| NIM 2D | AGTTGAGCAAGGCCAACTCGATTTTCAAAAT | SEQ ID NO:6 | Upstream |
| | T C A T GG | | |
| | T | | |

NIM1 homologue DNA fragments are amplified from wheat and rice using cDNA as template. Degenerate primer PCR is preferably performed with Ready-To-Go PCR Beads (Amersham, Piscataway, N.J.) in a GeneAmp PCR System 9700 (PE Applied Biosystems, Foster City, Calif.). 5 to 10 ng of cDNA is used in each reaction, with each primer at a final concentration of 0.8 μM. Preferable cycling parameters are as follows: 94° C. for 1 minute; 3 cycles of [94° C. for 30 seconds; 37° C. for 30 seconds; 72° C. for 2 minutes]; 35 cycles of [94° C. for 30 seconds; 60° C. for 30 seconds; 72° C. for 2 minutes]; 72° C. for 7 minutes; 4° C. hold. Reaction products are analyzed on 2% agarose gels and DNA fragments of the appropriate size are excised. DNA fragments are isolated from agarose bands using, for example, the Geneclean III Kit (BIO 101, Inc., Carlsbad, Calif.) and cloned using, for example, the TOPO TA Cloning Kit (Invitrogen Corporation, Carlsbad, Calif.). Plasmids are isolated using, for example, the CONCERT Rapid Plasmid Miniprep System (Life Technologies, Inc., Rockville, Md.) and sequenced by standard protocols.

Using primers 2B and 2D, two unique NIM1 homologue DNA fragments are amplified from the rice cDNA library (SEQ ID NO:7 and 9) and one unique NIM1 homologue DNA fragment is amplified from the wheat cDNA library (SEQ ID NO:11).

### Example 6

### Full-Length Monocotyledous NIM1 Homologue cDNA's

Corresponding cDNA sequences upstream and downstream from the NIM1 homologue PCR fragments are preferably obtained by RACE PCR using the SMART RACE cDNA Amplification Kit (Clontech, Palo Alto, Calif.). Preferably, at least three independent RACE products are sequenced for each 5′- or 3′-end in order to eliminate PCR errors. A full-length rice NIM1 homologue cDNA sequence corresponding to the PCR fragment shown in SEQ ID NO:7 is presented as SEQ ID NO:13; a NIM1 homologue rice cDNA sequence corresponding to the PCR fragment shown in SEQ ID NO:9 is presented as SEQ ID NO:15; and a full-length wheat NIM1 homologue cDNA sequence corresponding to the PCR fragment shown in SEQ ID NO:11 is presented as SEQ ID NO:17.

A full-length wheat NIM1 homologue cDNA sequence corresponding to the wheat NIM1 genomic sequence pHW01 (SEQ ID NO:1) is preferably obtained by RACE PCR and is presented as SEQ ID NO:19. (The 3′ end of SEQ ID NO:19 is from a cDNA prediction program.)

II. Expression of the Gene Sequences of the Invention in Plants

A monocotyledonous NIM1 homologue of the present invention can be incorporated into plant cells using conventional recombinant DNA technology. Generally, this involves inserting a coding sequence of the invention into an expression system to which the coding sequence is heterologous (i.e., not normally present) using standard cloning procedures known in the art. The vector contains the necessary elements for the transcription and translation of the inserted protein-coding sequences. A large number of vector systems known in the art can be used, such as plasmids, bacteriophage viruses and other modified viruses. Suitable vectors include, but are not limited to, viral vectors such as lambda vector systems λgtl1, λgtl0 and Charon 4; plasmid vectors such as pBl121, pBR322, pACYC177, pACYC184, pAR series, pKK223-3, pUC8, pUC9, pUC18, pUC19, pLG339, pRK290, pKC37, pKC101, pCDNAII; and other similar systems. The components of the expression system may also be modified to increase expression. For example, truncated sequences, nucleotide substitutions or other modifications may be employed. The expression systems described herein can be used to transform virtually any crop plant cell under suitable conditions. Transformed cells can be regenerated into whole plants such that the monocotyledonous NIM1 homologue plays a role in increasing SAR gene expression and enhancing disease resistance in the transgenic plants.

### Example 7

### Construction of Plant Expression Cassettes

Coding sequences intended for expression in transgenic plants are first assembled in expression cassettes behind a suitable promoter expressible in plants. The expression cassettes may also comprise any further sequences required or selected for the expression of the transgene. Such sequences include, but are not restricted to, transcription terminators, extraneous sequences to enhance expression such as introns, vital sequences, and sequences intended for the targeting of the gene product to specific organelles and cell compartments. These expression cassettes can then be easily transferred to the plant transformation vectors described below. The following is a description of various components of typical expression cassettes.

1. Promoters

The selection of the promoter used in expression cassettes will determine the spatial and temporal expression pattern of the transgene in the transgenic plant. Selected promoters will express transgenes in specific cell types (such as leaf epidermal cells, mesophyll cells, root cortex cells) or in specific tissues or organs (roots, leaves or flowers, for example) and the selection will reflect the desired location of accumulation of the gene product. Alternatively, the selected promoter may drive expression of the gene under various inducing conditions. Promoters vary in their strength, i.e., ability to promote transcription. Depending upon the host cell system utilized, any one of a number of suitable promoters can be used, including the gene's native promoter. The following are non-limiting examples of promoters that may be used in expression cassettes.

a. Constitutive Expression, the Ubiquitin Promoter:

Ubiquitin is a gene product known to accumulate in many cell types and its promoter has been cloned from several species for use in transgenic plants (e.g. sunflower—Binet et al.,1991; maize—Christensen et al., 1989; and Arabidopsis—Norris et al., 1993). The maize ubiquitin promoter has been developed in transgenic monocot systems and its sequence and vectors constructed for monocot transformation are disclosed in the patent publication EP 0 342 926 (to Lubrizol). Taylor et al. (1993) describe a vector (pAHC25) that comprises the maize ubiquitin promoter and first intron and its high activity in cell suspensions of numerous monocotyledons when introduced via microprojectile bombardment. The Arabidopsis ubiquitin promoter is especially preferred for use with the NIM1 homologues of the present invention. The ubiquitin promoter is suitable for gene expression in transgenic plants, both monocotyledons and dicotyledons. Suitable vectors are derivatives of pAHC25 or any of the transformation vectors described in this application, modified by the introduction of the appropriate ubiquitin promoter and/or intron sequences.

b. Constitutive Expression, the CaMV 35S Promoter:

Construction of the plasmid pCGN1761 is described in the published patent application EP 0 392 225 (Example 23). pCGN1761 contains the “double” CaMV 35S promoter and the tmI transcriptional terminator with a unique EcoRI site between the promoter and the terminator and has a pUC-type backbone. A derivative of pCGN1761 is constructed which has a modified polylinker which includes NotI and XhoI sites in addition to the existing EcoRI site. This derivative is designated pCGN1761ENX. pCGN1761ENX is useful for the cloning of cDNA sequences or coding sequences (including microbial ORF sequences) within its polylinker for the purpose of their expression under the control of the 35S promoter in transgenic plants. The entire 35S promoter-coding sequence-tmI terminator cassette of such a construction can be excised by HindIII, SphI, SaII, and XbaI sites 5′ to the promoter and XbaI, BamHI and BgII sites 3′ to the terminator for transfer to transformation vectors such as those described below. Furthermore, the double 35S promoter fragment can be removed by 5′ excision with HindIII, SphI, SaII, XbaI, or PstI, and 3′ excision with any of the polylinker restriction sites (EcoRI, NotI or XhoI) for replacement with another promoter. If desired, modifications around the cloning sites can be made by the introduction of sequences that may enhance translation. This is particularly useful when overexpression is desired. For example, pCGN1761 ENX may be modified by optimization of the translational initiation site as described in Example 37 of U.S. Pat. No. 5,639,949.

c. Constitutive Expression, the Actin Promoter:

Several isoforms of actin are known to be expressed in most cell types and consequently the actin promoter is a good choice for a constitutive promoter. In particular, the promoter from the rice ActI gene has been cloned and characterized (McElroy et al., 1990). A 1.3 kb fragment of the promoter was found to contain all the regulatory elements required for expression in rice protoplasts. Furthermore, numerous expression vectors based on the ActI promoter have been constructed specifically for use in monocotyledons (McElroy et al., 1991). These incorporate the ActI-intron 1, AdhI 5′ flanking sequence and AdhI-intron 1 (from the maize alcohol dehydrogenase gene) and sequence from the CamMV 35S promoter. Vectors showing highest expression were fusions of 35S and ActI intron or the ActI 5′ flanking sequence and the ActI intron. Optimization of sequences around the initiating ATG (of the GUS reporter gene) also enhanced expression. The promoter expression cassettes described by McElroy et al. (1991) can be easily modified for gene expression and are particularly suitable for use in monocotyledonous hosts. For example, promoter-containing fragments is removed from the McElroy constructions and used to replace the double 35S promoter in pCGN1761 ENX, which is then available for the insertion of specific gene sequences. The fusion genes thus constructed can then be transferred to appropriate transformation vectors. In a separate report, the rice ActI promoter with its first intron has also been found to direct high expression in cultured barley cells (Chibbar et al., 1993).

d. Inducible Expression, the PR-1 Promoter:

The double 35S promoter in pCGN1761 ENX may be replaced with any other promoter of choice that will result in suitably high expression levels. By way of example, one of the chemically regulatable promoters described in U.S. Pat. No. 5,614,395 may replace the double 35S promoter. The promoter of choice is preferably excised from its source by restriction enzymes, but can alternatively be PCR-amplified using primers that carry appropriate terminal restriction sites. Should PCR-amplification be undertaken, then the promoter should be re-sequenced to check for amplification errors after the cloning of the amplified promoter in the target vector. The chemically/pathogen regulatable tobacco PR-1a promoter is cleaved from plasmid pCIB1004 (for construction, see example 21 of EP 0 332 104) and transferred to plasmid pCGN1761 ENX (Uknes et al., 1992). pCIB1004 is cleaved with NcoI and the resultant 3′ overhang of the linearized fragment is rendered blunt by treatment with T4 DNA polymerase. The fragment is then cleaved with HindIII and the resultant PR-1a promoter-containing fragment is gel purified and cloned into pCGN1761 ENX from which the double 35S promoter has been removed. This is done by cleavage with XhoI and blunting with T4 polymerase, followed by cleavage with HindIII and isolation of the larger vector-terminator containing fragment into which the pCIB1004 promoter fragment is cloned. This generates a pCGN1761ENX derivative with the PR-1a promoter and the tmI terminator and an intervening polylinker with unique EcoRI and NotI sites. The selected coding sequence can be inserted into this vector, and the fusion products (i.e. promoter-gene-terminator) can subsequently be transferred to any selected transformation vector, including those described infra. Various chemical regulators may be employed to induce expression of the selected coding sequence in the plants transformed according to the present invention, including the benzothiadiazole, isonicotinic acid, and salicylic acid compounds disclosed in U.S. Pat. Nos. 5,523,311 and 5,614,395.

e. Inducible Expression, an Ethanol-Inducible Promoter:

A promoter inducible by certain alcohols or ketones, such as ethanol, may also be used to confer inducible expression of a coding sequence of the present invention. Such a promoter is for example the alcA gene promoter from Aspergillus nidulans (Caddick et al, 1998). In A. nidulans, the alcA gene encodes alcohol dehydrogenase I, the expression of which is regulated by the AlcR transcription factors in presence of the chemical inducer. For the purposes of the present invention, the CAT coding sequences in plasmid paIcA:CAT comprising a alcA gene promoter sequence fused to a minimal 35S promoter (Caddick et al., 1998) are replaced by a coding sequence of the present invention to form an expression cassette having the coding sequence under the control of the alcA gene promoter. This is carried out using methods well known in the art.

f. Inducible Expression, a Glucocorticoid-Inducible Promoter:

Induction of expression of a NIM1 homologue of the present invention using systems based on steroid hormones is also contemplated. For example, a glucocorticoid-mediated induction system is used (Aoyama and Chua, 1997) and gene expression is induced by application of a glucocorticoid, for example a synthetic glucocorticoid, preferably dexamethasone, preferably at a concentration ranging from 0.1 mM to 1 mM, more preferably from 10 mM to 100 mM. For the purposes of the present invention, the luciferase gene sequences are replaced by a gene sequence encoding a NIM1 homologue to form an expression cassette having the gene sequence encoding a NIM1 homologue under the control of six copies of the GAL4 upstream activating sequences fused to the 35S minimal promoter. This is carried out using methods well known in the art. The trans-acting factor comprises the GAL4 DNA-binding domain (Keegan et al., 1986) fused to the transactivating domain of the herpes viral protein VP16 (Triezenberg et al., 1988) fused to the hormone-binding domain of the rat glucocorticoid receptor (Picard et al., 1988). The expression of the fusion protein is controlled by any promoter suitable for expression in plants known in the art or described here. This expression cassette is also comprised in the plant comprising the gene sequence encoding a NIM1 homologue fused to the 6×GAL4/minimal promoter. Thus, tissue- or organ-specificity of the fusion protein is achieved leading to inducible tissue- or organ-specificity of the NIM1 homologue.

g. Root Specific Expression:

Another pattern of gene expression is root expression. A suitable root promoter is described by de Framond (1991) and also in the published patent application EP 0 452 269. This promoter is transferred to a suitable vector such as pCGN1761 ENX for the insertion of a selected gene and subsequent transfer of the entire promoter-gene-terminator cassette to a transformation vector of interest.

h. Wound-inducible Promoters:

Wound-inducible promoters may also be suitable for gene expression. Numerous such promoters have been described (e.g. Xu et al., 1993); Logemann et al., 1989; Rohrmeier & Lehle, 1993; Firek et al., 1993; Warner et al., 1993) and all are suitable for use with the instant invention. Logemann et al. describe the 5′ upstream sequences of the dicotyledonous potato wunI gene. Xu et al. show that a wound-inducible promoter from the dicotyledon potato (pin2) is active in the monocotyledon rice. Further, Rohrmeier & Lehle describe the cloning of the maize WipI cDNA which is wound induced and which can be used to isolate the cognate promoter using standard techniques. Similar, Firek et al. and Warner et al. have described a wound-induced gene from the monocotyledon Asparagus officinalis, which is expressed at local wound and pathogen invasion sites. Using cloning techniques well known in the art, these promoters can be transferred to suitable vectors, fused to the genes pertaining to this invention, and used to express these genes at the sites of plant wounding.

i. Pith-Preferred Expression:

Patent Application WO 93/07278 describes the isolation of the maize trpA gene, which is preferentially expressed in pith cells. The gene sequence and promoter extending up to −1726 bp from the start of transcription are presented. Using standard molecular biological techniques, this promoter, or parts thereof, can be transferred to a vector such as pCGN1761 where it can replace the 35S promoter and be used to drive the expression of a foreign gene in a pith-preferred manner. In fact, fragments containing the pith-preferred promoter or parts thereof can be transferred to any vector and modified for utility in transgenic plants.

j. Leaf-Specific Expression:

A maize gene encoding phosphoenol carboxylase (PEPC) has been described by Hudspeth & Grula (1989). Using standard molecular biological techniques the promoter for this gene can be used to drive the expression of any gene in a leaf-specific manner in transgenic plants.

k. Pollen-Specific Expression:

WO 93/07278 describes the isolation of the maize calcium-dependent protein kinase (CDPK) gene which is expressed in pollen cells. The gene sequence and promoter extend up to 1400 bp from the start of transcription. Using standard molecular biological techniques, this promoter or parts thereof, can be transferred to a vector such as pCGN1761 where it can replace the 35S promoter and be used to drive the expression of a NIM1 homologue of the present invention in a pollen-specific manner.

2. Transcriptional Terminators

A variety of transcriptional terminators are available for use in expression cassettes. These are responsible for the termination of transcription beyond the transgene and its correct polyadenylation. Appropriate transcriptional terminators are those that are known to function in plants and include the CaMV 35S terminator, the tmI terminator, the nopaline synthase terminator and the pea rbcS E9 terminator. These can be used in both monocotyledons and dicotyledons. In addition, a gene's native transcription terminator may be used.

3. Sequences for the Enhancement or Regulation of Expression

Numerous sequences have been found to enhance gene expression from within the transcriptional unit and these sequences can be used in conjunction with the genes of this invention to increase their expression in transgenic plants.

Various intron sequences have been shown to enhance expression, particularly in monocotyledonous cells. For example, the introns of the maize AdhI gene have been found to significantly enhance the expression of the wild-type gene under its cognate promoter when introduced into maize cells. Intron 1 was found to be particularly effective and enhanced expression in fusion constructs with the chloramphenicol acetyltransferase gene (Callis et al., 1987). In the same experimental system, the intron from the maize bronze1 gene had a similar effect in enhancing expression. Intron sequences have been routinely incorporated into plant transformation vectors, typically within the non-translated leader.

A number of non-translated leader sequences derived from viruses are also known to enhance expression, and these are particularly effective in dicotyledonous cells. Specifically, leader sequences from Tobacco Mosaic Virus (TMV, the “W-sequence”), Maize Chlorotic Mottle Virus (MCMV), and Alfalfa Mosaic Virus (AMV) have been shown to be effective in enhancing expression (e.g. Gallie et al., 1987; Skuzeski et al., 1990).

4. Targeting of the Gene Product Within the Cell

Various mechanisms for targeting gene products are known to exist in plants and the sequences controlling the functioning of these mechanisms have been characterized in some detail. For example, the targeting of gene products to the chloroplast is controlled by a signal sequence found at the amino terminal end of various proteins which is cleaved during chloroplast import to yield the mature protein (e.g. Comai et al., 1988). These signal sequences can be fused to heterologous gene products to effect the import of heterologous products into the chloroplast (van den Broeck, et al., 1985). DNA encoding for appropriate signal sequences can be isolated from the 5′ end of the cDNAs encoding the RUBISCO protein, the CAB protein, the EPSP synthase enzyme, the GS2 protein and many other proteins which are known to be chloroplast localized. See also, the section entitled “Expression With Chloroplast Targeting” in Example 37 of U.S. Pat. No. 5,639,949.

Other gene products are localized to other organelles such as the mitochondrion and the peroxisome (e.g. Unger et al., 1989). The cDNAs encoding these products can also be manipulated to effect the targeting of heterologous gene products to these organelles. Examples of such sequences are the nuclear-encoded ATPases and specific aspartate amino transferase isoforms for mitochondria. Targeting cellular protein bodies has been described by Rogers et al. (1985).

In addition, sequences have been characterized which cause the targeting of gene products to other cell compartments. Amino terminal sequences are responsible for targeting to the ER, the apoplast, and extracellular secretion from aleurone cells (Koehler & Ho, 1990). Additionally, amino terminal sequences in conjunction with carboxy terminal sequences are responsible for vacuolar targeting of gene products (Shinshi et al., 1990).

By the fusion of the appropriate targeting sequences described above to transgene sequences of interest it is possible to direct the transgene product to any organelle or cell compartment. For chloroplast targeting, for example, the chloroplast signal sequence from the RUBISCO gene, the CAB gene, the EPSP synthase gene, or the GS2 gene is fused in frame to the amino terminal ATG of the transgene. The signal sequence selected should include the known cleavage site, and the fusion constructed should take into account any amino acids after the cleavage site which are required for cleavage. In some cases this requirement may be fulfilled by the addition of a small number of amino acids between the cleavage site and the transgene ATG or, alternatively, replacement of some amino acids within the transgene sequence. Fusions constructed for chloroplast import can be tested for efficacy of chloroplast uptake by in vitro translation of in vitro transcribed constructions followed by in vitro chloroplast uptake using techniques described by Bartlett et al. (1982) and Wasmann et al. (1986). These construction techniques are well known in the art and are equally applicable to mitochondria and peroxisomes.

The above-described mechanisms for cellular targeting can be utilized not only in conjunction with their cognate promoters, but also in conjunction with heterologous promoters so as to effect a specific cell-targeting goal under the transcriptional regulation of a promoter that has an expression pattern different to that of the promoter from which the targeting signal derives.

### Example 8

### Construction of Plant Transformation Vectors

Numerous transformation vectors available for plant transformation are known to those of ordinary skill in the plant transformation arts, and the genes pertinent to this invention can be used in conjunction with any such vectors. The selection of vector will depend upon the preferred transformation technique and the target species for transformation. For certain target species, different antibiotic or herbicide selection markers may be preferred. Selection markers used routinely in transformation include the nptII gene, which confers resistance to kanamycin and related antibiotics (Messing & Vierra, 1982; Bevan et al., 1983), the bargene, which confers resistance to the herbicide phosphinothricin (White et al., 1990; Spencer et al., 1990), the hph gene, which confers resistance to the antibiotic hygromycin (Blochinger & Diggelmann), and the dhfr gene, which confers resistance to methatrexate (Bourouis et al., 1983), and the EPSPS gene, which confers resistance to glyphosate (U.S. Pat. Nos. 4,940,935 and 5,188,642).

1. Vectors Suitable for Agrobacterium Transformation

Many vectors are available for transformation using Agrobacterium tumefaciens. These typically carry at least one T-DNA border sequence and include vectors such as pBIN19 (Bevan, Nucl. Acids Res. (1984)) and pXYZ. Below, the construction of two typical vectors suitable for Agrobacterium transformation is described.

a. pCIB200 and pCIB2001:

The binary vectors pcIB200 and pCIB2001 are used for the construction of recombinant vectors for use with Agrobacterium and are constructed in the following manner. pTJS75kan is created by NarI digestion of pTJS75 (Schmidhauser & Helinski, 1985) allowing excision of the tetracycline-resistance gene, followed by insertion of an AccI fragment from pUC4K carrying an NPTII (Messing & Vierra, 1982; Bevan et al., 1983; McBride et al., 1990). XhoI linkers are ligated to the EcoRV fragment of PCIB7 which contains the left and right T-DNA borders, a plant selectable nos/nptII chimeric gene and the pUC polylinker (Rothstein et al., 1987), and the XhoI-digested fragment are cloned into SaII-digested pTJS75kan to create pCIB200 (see also EP 0 332 104, example 19). pCIB200 contains the following unique polylinker restriction sites: EcoRI, SstI, KpnI, BgIII, XbaI, and SaII. pCIB2001 is a derivative of pCIB200 created by the insertion into the polylinker of additional restriction sites. Unique restriction sites in the polylinker of pCIB2001 are EcoRI, SstI, KpnI, BgIII, XbaI, SaII, MluI, BcII, AvrII, ApaI, HpaI, and StuI. pCIB2001, in addition to containing these unique restriction sites also has plant and bacterial kanamycin selection, left and right T-DNA borders for Agrobacterium-mediated transformation, the RK2-derived trfA function for mobilization between E. coli and other hosts, and the OriT and OriV functions also from RK2. The pCIB2001 polylinker is suitable for the cloning of plant expression cassettes containing their own regulatory signals.

b. pCIB10 and Hygromycin Selection Derivatives Thereof:

The binary vector pCIB10 contains a gene encoding kanamycin resistance for selection in plants and T-DNA right and left border sequences and incorporates sequences from the wide host-range plasmid pRK252 allowing it to replicate in both E. coli and Agrobacterium. Its construction is described by Rothstein et al. (1987). Various derivatives of pCIB10 are constructed which incorporate the gene for hygromycin B phosphotransferase described by Gritz et al., 1983). These derivatives enable selection of transgenic plant cells on hygromycin only (pCIB743), or hygromycin and kanamycin (pCIB715, pCIB717).

2. Vectors Suitable for non-Agrobacterium Transformation

Transformation without the use of Agrobacterium tumefaciens circumvents the requirement for T-DNA sequences in the chosen transformation vector and consequently vectors lacking these sequences can be utilized in addition to vectors such as the ones described above which contain T-DNA sequences. Transformation techniques that do not rely on Agrobacterium include transformation via particle bombardment, protoplast uptake (e.g. PEG and electroporation) and microinjection. The choice of vector depends largely on the preferred selection for the species being transformed. Below, the construction of typical vectors suitable for non-Agrobacterium transformation is described.

a. pCIB3064:

pCIB3064 is a pUC-derived vector suitable for direct gene transfer techniques in combination with selection by the herbicide basta (or phosphinothricin). The plasmid pCIB246 comprises the CaMV 35S promoter in operational fusion to the E. coli GUS gene and the CaMV 35S transcriptional terminator and is described in the PCT published application WO 93/07278. The 35S promoter of this vector contains two ATG sequences 5′ of the start site. These sites are mutated using standard PCR techniques in such a way as to remove the ATGs and generate the restriction sites SspI and PvuII. The new restriction sites are 96 and 37 bp away from the unique SaII site and 101 and 42 bp away from the actual start site. The resultant derivative of pCIB246 is designated pCIB3025. The GUS gene is then excised from pCIB3025 by digestion with SaII and SacI, the termini rendered blunt and religated to generate plasmid pCIB3060. The plasmid pJIT82 is obtained from the John Innes Centre, Norwich and the a 400 bp SmaI fragment containing the bar gene from Streptomyces viridochromogenes is excised and inserted into the HpaI site of pCIB3060 (Thompson et al., 1987). This generated pCIB3064, which comprises the bargene under the control of the CaMV 35S promoter and terminator for herbicide selection, a gene for ampicillin resistance (for selection in E. coli) and a polylinker with the unique sites SphI, PstI, HindIII, and BamHI. This vector is suitable for the cloning of plant expression cassettes containing their own regulatory signals.

b. pSOG19 and pSOG35:

pSOG35 is a transformation vector that utilizes the E. coli gene dihydrofolate reductase (DFR) as a selectable marker conferring resistance to methotrexate. PCR is used to amplify the 35S promoter (−800 bp), intron 6 from the maize Adh1 gene (−550 bp) and 18 bp of the GUS untranslated leader sequence from pSOG10. A 250-bp fragment encoding the E. coli dihydrofolate reductase type II gene is also amplified by PCR and these two PCR fragments are assembled with a SacI-PstI fragment from pB1221 (Clontech) which comprises the pUC19 vector backbone and the nopaline synthase terminator. Assembly of these fragments generates pSOG19 which contains the 35S promoter in fusion with the intron 6 sequence, the GUS leader, the DHFR gene and the nopaline synthase terminator. Replacement of the GUS leader in pSOG19 with the leader sequence from Maize Chlorotic Mottle Virus (MCMV) generates the vector pSOG35. pSOG19 and pSOG35 carry the pUC gene for ampicillin resistance and have HindIII, SphI, PstI and EcoRI sites available for the cloning of foreign substances.

### Example 9

### Transformation

Once the gene sequence of interest has been cloned into an expression system, it is transformed into a plant cell. Methods for transformation and regeneration of plants are well known in the art. For example, Ti plasmid vectors have been utilized for the delivery of foreign DNA, as well as direct DNA uptake, liposomes, electroporation, micro-injection, and microprojectiles. In addition, bacteria from the genus Agrobacterium can be utilized to transform plant cells. Below are descriptions of representative techniques for transforming both dicotyledonous and monocotyledonous plants.

1. Transformation of Dicotyledons

Transformation techniques for dicotyledons are well known in the art and include Agrobacterium based techniques and techniques that do not require Agrobacterium. Non-Agrobacterium techniques involve the uptake of exogenous genetic material directly by protoplasts or cells. This can be accomplished by PEG or electroporation mediated uptake, particle bombardment-mediated delivery, or microinjection. Examples of these techniques are described by Paszkowski et al., 1984; Potrykus et al., 1985; Reich et al., 1986; and Klein et al., 1987. In each case the transformed cells are regenerated to whole plants using standard techniques known in the art.

Agrobacterium-mediated transformation is a preferred technique for transformation of dicotyledons because of its high efficiency of transformation and its broad utility with many different species. Agrobacterium transformation typically involves the transfer of the binary vector carrying the foreign DNA of interest (e.g. pCIB200 or pCIB2001) to an appropriate Agrobacterium strain which may depend of the complement of vir genes carried by the host Agrobacterium strain either on a co-resident Ti plasmid or chromosomally (e.g. strain CIB542 for pCIB200 and pCIB2001 (Uknes et al., 1993). The transfer of the recombinant binary vector to Agrobacterium is accomplished by a triparental mating procedure using E. coli carrying the recombinant binary vector, a helper E. coli strain which carries a plasmid such as pRK2013 and which is able to mobilize the recombinant binary vector to the target Agrobacterium strain. Alternatively, the recombinant binary vector can be transferred to Agrobacterium by DNA transformation (Höfgen & Willmitzer, 1988).

Transformation of the target plant species by recombinant Agrobacterium usually involves co-cultivation of the Agrobacterium with explants from the plant and follows protocols well known in the art. Transformed tissue is regenerated on selectable medium carrying the antibiotic or herbicide resistance marker present between the binary plasmid T-DNA borders.

Another approach to transforming plant cells with a gene involves propelling inert or biologically active particles at plant tissues and cells. This technique is disclosed in U.S. Pat. Nos. 4,945,050, 5,036,006, and 5,100,792. Generally, this procedure involves propelling inert or biologically active particles at the cells under conditions effective to penetrate the outer surface of the cell and afford incorporation within the interior thereof. When inert particles are utilized, the vector can be introduced into the cell by coating the particles with the vector containing the desired gene. Alternatively, the target cell can be surrounded by the vector so that the vector is carried into the cell by the wake of the particle. Biologically active particles (e.g., dried yeast cells, dried bacterium or a bacteriophage, each containing DNA sought to be introduced) can also be propelled into plant cell tissue.

2. Transformation of Monocotyledons

Transformation of most monocotyledon species has now also become routine. Preferred techniques include direct gene transfer into protoplasts using PEG or electroporation techniques, and particle bombardment into callus tissue. Transformations can be undertaken with a single DNA species or multiple DNA species (i.e. co-transformation) and both these techniques are suitable for use with this invention. Co-transformation may have the advantage of avoiding complete vector construction and of generating transgenic plants with unlinked loci for the gene of interest and the selectable marker, enabling the removal of the selectable marker in subsequent generations, should this be regarded desirable. However, a disadvantage of the use of co-transformation is the less than 100% frequency with which separate DNA species are integrated into the genome (Schocher et al., 1986).

Patent Applications EP 0 292 435, EP 0 392 225, and WO 93/07278 describe techniques for the preparation of callus and protoplasts from an elite inbred line of maize, transformation of protoplasts using PEG or electroporation, and the regeneration of maize plants from transformed protoplasts. Gordon-Kamm et al. (1990) and Fromm et al. (1990) have published techniques for transformation of Al 88-derived maize line using particle bombardment. Furthermore, WO 93/07278 and Koziel et al. (1993) describe techniques for the transformation of elite inbred lines of maize by particle bombardment. This technique utilizes immature maize embryos of 1.5-2.5 mm length excised from a maize ear 14-15 days after pollination and a PDS-100He Biolistics device for bombardment.

Transformation of rice can also be undertaken by direct gene transfer techniques utilizing protoplasts or particle bombardment. Protoplast-mediated transformation has been described for Japonica-types and Indica-types (Zhang et al., 1988; Shimamoto et al., 1989; Datta et al., 1990). Both types are also routinely transformable using particle bombardment (Christou et al., 1991). Furthermore, WO 93/21335 describes techniques for the transformation of rice via electroporation.

Patent Application EP 0 332 581 describes techniques for the generation, transformation and regeneration of Pooideae protoplasts. These techniques allow the transformation of Dactylis and wheat. Furthermore, wheat transformation has been described by Vasil et al. (1992) using particle bombardment into cells of type C long-term regenerable callus, and also by Vasil et al. (1993) and Weeks et al. (1993) using particle bombardment of immature embryos and immature embryo-derived callus. A preferred technique for wheat transformation, however, involves the transformation of wheat by particle bombardment of immature embryos and includes either a high sucrose or a high maltose step prior to gene delivery. Prior to bombardment, any number of embryos (0.75-1 mm in length) are plated onto MS medium with 3% sucrose (Murashiga & Skoog, 1962) and 3 mg/l 2.4-D for induction of somatic embryos, which is allowed to proceed in the dark. On the chosen day of bombardment, embryos are removed from the induction medium and placed onto the osmoticum (i.e. induction medium with sucrose or maltose added at the desired concentration, typically 15%). The embryos are allowed to plasmolyze for 2-3 h and are then bombarded. Twenty embryos per target plate is typical, although not critical. An appropriate gene-carrying plasmid (such as pCIB3064 or pSG35) is precipitated onto micrometer size gold particles using standard procedures. Each plate of embryos is shot with the DuPont Biolistics® helium device using a burst pressure of ˜1000 psi using a standard 80 mesh screen. After bombardment, the embryos are placed back into the dark to recover for about 24 h (still on osmoticum). After 24 hrs, the embryos are removed from the osmoticum and placed back onto induction medium where they stay for about a month before regeneration. Approximately one month later the embryo explants with developing embryogenic callus are transferred to regeneration medium (MS+1 mg/liter NM, 5 mg/liter GA), further containing the appropriate selection agent (10 mg/l basta in the case of pCIB3064 and 2 mg/l methotrexate in the case of pSOG35). After approximately one month, developed shoots are transferred to larger sterile containers known as “GA7s” which contain half-strength MS, 2% sucrose, and the same concentration of selection agent.

Tranformation of monocotyledons using Agrobacterium has also been described. See, WO 94/00977 and U.S. Pat. No. 5,591,616.

III. Breeding and Seed Production

### Example 10

### Breeding

The plants obtained via tranformation with a gene of the present invention can be any of a wide variety of plant species, including those of monocots and dicots; however, the plants used in the method of the invention are preferably selected from the list of agronomically important target crops set forth supra. The expression of a gene of the present invention in combination with other characteristics important for production and quality can be incorporated into plant lines through breeding. Breeding approaches and techniques are known in the art. See, for example, Welsh J. R. (1981); Wood D. R. (Ed.) (1983); Mayo O. (1987); Singh, D. P. (1986); and Wricke and Weber (1986).

The genetic properties engineered into the transgenic seeds and plants described above are passed on by sexual reproduction or vegetative growth and can thus be maintained and propagated in progeny plants. Generally said maintenance and propagation make use of known agricultural methods developed to fit specific purposes such as tilling, sowing or harvesting. Specialized processes such as hydroponics or greenhouse technologies can also be applied. As the growing crop is vulnerable to attack and damages caused by insects or infections as well as to competition by weed plants, measures are undertaken to control weeds, plant diseases, insects, nematodes, and other adverse conditions to improve yield. These include mechanical measures such a tillage of the soil or removal of weeds and infected plants, as well as the application of agrochemicals such as herbicides, fungicides, gametocides, nematicides, growth regulants, ripening agents and insecticides.

Use of the advantageous genetic properties of the transgenic plants and seeds according to the invention can further be made in plant breeding, which aims at the development of plants with improved properties such as tolerance of pests, herbicides, or stress, improved nutritional value, increased yield, or improved structure causing less loss from lodging or shattering. The various breeding steps are characterized by well-defined human intervention such as selecting the lines to be crossed, directing pollination of the parental lines, or selecting appropriate progeny plants. Depending on the desired properties, different breeding measures are taken. The relevant techniques are well known in the art and include but are not limited to hybridization, inbreeding, backcross breeding, multiline breeding, variety blend, interspecific hybridization, aneuploid techniques, etc. Hybridization techniques also include the sterilization of plants to yield male or female sterile plants by mechanical, chemical, or biochemical means. Cross pollination of a male sterile plant with pollen of a different line assures that the genome of the male sterile but female fertile plant will uniformly obtain properties of both parental lines. Thus, the transgenic seeds and plants according to the invention can be used for the breeding of improved plant lines, that for example, increase the effectiveness of conventional methods such as herbicide or pestidice treatment or allow one to dispense with said methods due to their modified genetic properties. Alternatively new crops with improved stress tolerance can be obtained, which, due to their optimized genetic “equipment”, yield harvested product of better quality than products that were not able to tolerate comparable adverse developmental conditions.

### Example 11

### Seed Production

In seeds production, germination quality and uniformity of seeds are essential product characteristics, whereas germination quality and uniformity of seeds harvested and sold by the farmer is not important. As it is difficult to keep a crop free from other crop and weed seeds, to control seedborne diseases, and to produce seed with good germination, fairly extensive and well-defined seed production practices have been developed by seed producers, who are experienced in the art of growing, conditioning and marketing of pure seed. Thus, it is common practice for the farmer to buy certified seed meeting specific quality standards instead of using seed harvested from his own crop. Propagation material to be used as seeds is customarily treated with a protectant coating comprising herbicides, insecticides, fungicides, bactericides, nematicides, molluscicides, or mixtures thereof. Customarily used protectant coatings comprise compounds such as captan, carboxin, thiram (TMTD®), methalaxyl (Apron®), and pirimiphos-methyl (Actellic®). If desired, these compounds are formulated together with further carriers, surfactants or application-promoting adjuvants customarily employed in the art of formulation to provide protection against damage caused by bacterial, fungal or animal pests. The protectant coatings may be applied by impregnating propagation material with a liquid formulation or by coating with a combined wet or dry formulation. Other methods of application are also possible such as treatment directed at the buds or the fruit.

It is a further aspect of the present invention to provide new agricultural methods, such as the methods examplified above, which are characterized by the use of transgenic plants, transgenic plant material, or transgenic seed according to the present invention.

The seeds may be provided in a bag, container or vessel comprised of a suitable packaging material, the bag or container capable of being closed to contain seeds. The bag, container or vessel may be designed for either short term or long term storage, or both, of the seed. Examples of a suitable packaging material include paper, such as kraft paper, rigid or pliable plastic or other polymeric material, glass or metal. Desirably the bag, container, or vessel is comprised of a plurality of layers of packaging materials, of the same or differing type. In one embodiment the bag, container or vessel is provided so as to exclude or limit water and moisture from contacting the seed. In one example, the bag, container or vessel is sealed, for example heat sealed, to prevent water or moisture from entering. In another embodiment water absorbent materials are placed between or adjacent to packaging material layers. In yet another embodiment the bag, container or vessel, or packaging material of which it is comprised is treated to limit, suppress or prevent disease, contamination or other adverse affects of storage or transport of the seed. An example of such treatment is sterilization, for example by chemical means or by exposure to radiation. Comprised by the present invention is a commercial bag comprising seed of a transgenic plant comprising a gene of the present invention that is expressed in said transformed plant at higher levels than in a wild type plant, together with a suitable carrier, together with label instructions for the use thereof for conferring broad spectrum disease resistance to plants.

IV. Disease Resistance Evaluation

Disease resistance evaluation is performed by methods known in the art. See, Uknes et al. (1993); Görlach et al. (1996); Alexander et al. (1993). For example, several representative disease resistance assays are described below.

### Example 12

### Phytophthora parasitica (Black Shank) Resistance Assay

Assays for resistance to Phytophthora parasitica, the causative organism of black shank, are performed on six-week-old plants grown as described in Alexander et al. (1993). Plants are watered, allowed to drain well, and then inoculated by applying 10 ml of a sporangium suspension (300 sporangia/ml) to the soil. Inoculated plants are kept in a greenhouse maintained at 23-25° C. day temperature, and 20-22° C. night temperature. The wilt index used for the assay is as follows: 0=no symptoms; 1=no symptoms; 1=some sign of wilting, with reduced turgidity; 2=clear wilting symptoms, but no rotting or stunting; 3=clear wilting symptoms with stunting, but no apparent stem rot; 4=severe wilting, with visible stem rot and some damage to root system; 5=as for 4, but plants near death or dead, and with severe reduction of root system. All assays are scored blind on plants arrayed in a random design.

### Example 13

### Pseudomonas syringae Resistance Assay

Pseudomonas syringae pv. tabaci strain #551 is injected into the two lower leaves of several 6-7-week-old plants at a concentration of 10⁶ or 3×10⁶ per ml in H₂0. Six individual plants are evaluated at each time point. Pseudomonas tabaci infected plants are rated on a 5 point disease severity scale, 5=100% dead tissue, 0=no symptoms. A T-test (LSD) is conducted on the evaluations for each day and the groupings are indicated after the Mean disease rating value. Values followed by the same letter on that day of evaluation are not statistically significantly different.

### Example 14

### Cercospora nicotianae Resistance Assay

A spore suspension of Cercospora nicotianae (ATCC #18366) (100,000-150,000 spores per ml) is sprayed to imminent run-off onto the surface of the leaves. The plants are maintained in 100% humidity for five days. Thereafter the plants are misted with water 5-10 times per day. Six individual plants are evaluated at each time point. Cercospora nicotianae is rated on a % leaf area showing disease symptoms basis. A T-test (LSD) is conducted on the evaluations for each day and the groupings are indicated after the Mean disease rating value. Values followed by the same letter on that day of evaluation are not statistically significantly different.

### Example 15

### Peronospora parasitica Resistance Assay

Assays for resistance to Peronospora parasitica are performed on plants as described in Uknes et al, (1993). Plants are inoculated with a compatible isolate of P. parasitica by spraying with a conidial suspension (approximately 5×10⁴ spores per milliliter). Inoculated plants are incubated under humid conditions at 17° C. in a growth chamber with a 14-hr day/10-hr night cycle. Plants are examined at 3-14 days, preferably 7-12 days, after inoculation for the presence of conidiophores. In addition, several plants from each treatment are randomly selected and stained with lactophenol-trypan blue (Keogh et al., 1980) for microscopic examination.

The above disclosed embodiments are illustrative. This disclosure of the invention will place one skilled in the art in possession of many variations of the invention. All such obvious and foreseeable variations are intended to be encompassed by the claims.

1. An isolated nucleic acid molecule comprising a nucleotide sequence from a monocotyledonous plant that is a homologue of the NIM1 gene.

2. An isolated nucleic acid molecule according to claim 1, comprising:
(a) a nucleotide sequence that encodes SEQ ID NO:2, 8, 10, 12, 14,16, 18, or 20;
(b) SEQ ID NO:1, 7, 9, 11, 13, 15, 17, or 19;
(c) a nucleotide sequence that comprises an at least 20 consecutive base pair portion identical in sequence to an at least 20 consecutive base pair portion of SEQ ID NO:1, 7, 9, 11, 13, 15, 17, or 19;
(d) a nucleotide sequence that can be amplified from a monocotyledonous plant DNA library using the polymerase chain reaction with the pair of primers set forth as SEQ ID NO:3 and 4 or SEQ ID NO:5 and 6;
(e) a nucleotide sequence that can be amplified from a Orzya sativa DNA library using the polymerase chain reaction with the pair of primers set forth as SEQ ID NO:3 and 4 or SEQ ID NO:5 and 6;
(f) a nucleotide sequence that can be amplified from a Triticum aestivum DNA library using the polymerase chain reaction with the pair of primers set forth as SEQ ID NO:3 and 4 or SEQ ID NO:5 and 6;
(g) a nucleotide sequence that can be amplified from a monocotyledonous plant DNA library using the polymerase chain reaction with a pair of primers comprising the first 20 nucleotides and the reverse complement of the last 20 nucleotides of the coding sequence (CDS) of SEQ ID NO:1, 7, 9, 11, 13, 15, 17, or 19; or
(h) a nucleotide sequence that hybridizes to the complement of SEQ ID NO:1, 7, 9, 11, 13, 15, 17, or 19 under stringent hybridization and wash conditions.

3. An isolated nucleic acid molecule according to claim 2, comprising a nucleotide sequence that encodes SEQ ID NO:2, 8, 10, 12, 14, 16, 18, or 20.

4. An isolated nucleic acid molecule according to claim 2, comprising SEQ ID NO:1, 7, 9, 11, 13, 15, 17, or 19.

5. An isolated nucleic acid molecule according to claim 2, comprising a nucleotide sequence that comprises an at least 20 consecutive base pair portion identical in sequence to an at least 20 consecutive base pair portion of SEQ ID NO:1, 7, 9, 11, 13, 15, 17, or 19.

6. An isolated nucleic acid molecule according to claim 2, comprising a nucleotide sequence that can be amplified from a monocotyledonous plant DNA library using the polymerase chain reaction with the pair of primers set forth as SEQ ID NO:3 and 4 or SEQ ID NO:5 and 6.

7. An isolated nucleic acid molecule according to claim 2, comprising a nucleotide sequence that can be amplified from a Orzya sativa DNA library using the polymerase chain reaction with the pair of primers set forth as SEQ ID NO:3 and 4 or SEQ ID NO:5 and 6.

8. An isolated nucleic acid molecule according to claim 2, comprising a nucleotide sequence that can be amplified from a Triticum aestivum DNA library using the polymerase chain reaction with the pair of primers set forth as SEQ ID NO:3 and 4 or SEQ ID NO:5 and 6.

9. An isolated nucleic acid molecule according to claim 2, comprising a nucleotide sequence that can be amplified from a monocotyledonous plant DNA library using the polymerase chain reaction with a pair of primers corresponding to the first 20 nucleotides and the reverse complement of the last 20 nucleotides of the coding sequence (CDS) of SEQ ID NO:1, 7, 9, 11, 13, 15, 17, or 19.

10. An isolated nucleic acid molecule according to claim 2, comprising a nucleotide sequence that hybridizes to the complement of SEQ ID NO:1, 7, 9, 11, 13, 15, 17, or 19 under stringent hybridization and wash conditions.

11. A chimeric gene comprising a promoter active in plants operatively linked to the nucleic acid molecule of claim 1.

12. A recombinant vector comprising the chimeric gene of claim 11.

13. A host cell comprising the chimeric gene of claim 11.

14. A plant comprising the chimeric gene of claim 13.

15. The plant of claim 14, which is a monocotyledonous plant.

16. The plant of claim 14, which is selected from the following: rice, wheat, barley, rye, corn, potato, canola, sunflower, carrot, sweet potato, sugarbeet, bean, pea, chicory, lettuce, cabbage, cauliflower, broccoli, turnip, radish, spinach, asparagus, onion, garlic, eggplant, pepper, celery, squash, pumpkin, cucumber, apple, pear, quince, melon, plum, cherry, peach, nectarine, apricot, strawberry, grape, raspberry, blackberry, pineapple, avocado, papaya, mango, banana, soybean, tobacco, tomato, sorghum and sugarcane.

17. Seed from the plant of claim 14.

18. A method of increasing SAR gene expression in a plant, comprising expressing the chimeric gene of claim 11 in said plant.

19. A method of enhancing disease resistance in a plant, comprising expressing the chimeric gene of claim 11 in said plant.

20. A PCR primer that is SEQ ID NO:3 or SEQ ID NO:4.

21. A method for isolating a NIM1 homologue involved in the signal transduction cascade leading to systemic acquired resistance in plants comprising amplifying a DNA molecule from a plant DNA library using the polymerase chain reaction with a pair of primers corresponding to the first 20 nucleotides and the reverse complement of the last 20 nucleotides of the coding sequence (CDS) of SEQ ID NO:1, 7, 9, 11, 13, 15, 17, or 19 or with the pair of primers set forth as SEQ ID NO:3 and 4 or SEQ ID NO:5 and 6.

22. The method of claim 21, wherein said plant DNA library is a Oryza sativa (rice) or Triticum aestivum (wheat) DNA library.


SEQUENCE LISTING (20 sequences)

SEQ ID NO: 1 - DNA, 4270 bp, Triticum aestivum
  exon (1396)..(2163)
  intron (2164)..(2337)
  exon (2338)..(2532)
  intron (2533)..(2933)
  exon (2934)..(3188)
gagctcgccaacctcttccaggtccgcctctccctctccccttctcctccatgatgctttcttggtttcagacatttattgtgcttgctgggaatgcatatttgcgcgcacgttcttgtgctcagacagcaaggtttaatgctgtcttttctttctgcacgcggggacgttttctgtatgcggcaaaatgggcttagatccccttaccatttctgctaaatttaatcaatttcagtacttctgaaaaatagcgttaaacattggttagtactagtacgttttgtcggtagcaatgaggagcttgtgcttatcatgtggtgatcttgaaattggtgaagttgtcaatggaaattgtacagttgggaccttgaggtgccgtgtcattttgatgctatctcaaggattcttgttctgatgttttttttttcttggggaaaaatggtaattgttcattgctcaaagaatgagtggtgtcaatatggtacatgcccctacttatatttttcatcaatgaaatgcagttcttatgaaactgtacaaatctaggttgcattaatgcagacgtttggtacatatacaatacaaaggaaagcatgtacagcacctttcccccggatacaataggaaagcatgtgcaccacctttccccagacaattcacaacaccgggagtctgcgacagtattatatcgtctgttttctcacttaataaagtttcgggtgtcagtgtgtaaagcgcctaatattcctaatgttcataaacatatttgctccacaactccttaatttccattaggatcatctattaatgttattctgagcaggagtgttttgatagtgaacgccctttttaggtaaccagtcatcaatccatggatctgtgcatgatataaatgtttagatggaggttcactgtaaaagattttataaccaatttgtattctaatgtacttgcgagcatttgaaacacataaaattacttttgataggttacttaaatatatgcaacttcgatgcagaggctggggtaataaaatcttccattttctattttttgaaatacttgttgacagggctgtaatcaaattgggttaatcaatgtatgtgtttgtattcttaaaatattacttatcagattagaccgtttatgcgtctatattcttatcaatccgtatggctgtgtcgagacttcggatttttatgtattttttagtgatgatatgcttttccttcttagctttgtcatactgagatttgtgttttaataattctgacttcgctgcagatgatttgcccgtgtatcgtttgatgctaactctcgtcgacttgctacttgtaacagttctctattgttctattgtttcatgtttttgagaagcgagtactaacccatgttatgcccttcttttccatgcagcggcatctccttgatttccttgataaagttgaagtggataaccttccgttgatcttatctgttgcaaacttatgcaacaaatcttgcgtgaaactgttcgagagatgcatggagatggtagtccggtcaaatcttgacatgattactctagagaaagcattgcctcaagatgtcatcaagcaaattactgatttacggataactcttggattagcttcacccgaagacaatggctttcctaacaaacacgtaagaaggatactcagagcacttgattctgatgatgtggagcttgtcaggatgctgctcacagaagggcagactaaccttgatgatgcatttgcattgcactatgctgtagaacactgtgactcaaaaattacaacagaacttctggacatcgcacttgcggatgttaatctcagaaacccaagaggttatactgttcttcacatcgccgctaagcggagagatcctaaaatcgttgtctcccttttaaccaaaggtgcccggccttcagattttacatttgatggaagaaaagcagttcaaatctcaaagagactcacaaaacatggtgattattttgggaatactgaagaaggaaagccgtctcccaatgataaattatgcattgagatattggagcaagctgaaagaagggatccacaacttggagaagcatcactttctcttgcattggctggtgactgtcttcgtggaaagttactgtaccttgaaaaccgaggtaatattcatctacatatggtgtagtgttcaaaattagttgtttggttggatttacagttggtggtgttttgcatgtgaagtgaagctttccacgggcaattagcaatgtgttaaccttttttttattctcttggaagttgtgttttcacacaattattttcatgctttgtagttgctttggcaaggataatgtttccaattgaggcaagagtagcaatggacattgctcaagtggatggtactttggaatttacccttggttctagtacaaatccacctctggagataacaaccgttgatctgaatgatacttctttcaaaatgaaggaggaacacttagctcggatgagagccctctccaaaacaggtgaggcacagcttgcgtttttatcttttggttgttctgtttttagctttctagatttttgttcctgccttttcctttttgtgttagtttgcttgtaagacccgttccatgttagcgatttcttctggtacaaatgacgcagacttggggggtgtgctcaagaaagaaagaaaaatatcaataggattactggtgtagataaggttgaaatcccagattttattctctctcctgttaagaactgaagagtgaacccaaatatgtacacatacgaaatgttaattggtggtcgaattgctaatgattactctcgcatgtgtgtgcgcgtgcattcagctataagatgacattttaccaagcttatgtatctggactaactgcaatctgaatgtttataccagttgaactcggcaaacgtttcttcccacgctgttcaaatgtgctggacaagatcatggacgatgaacctgagctggcttccctcggaagagatgcatcctccgagaggaagaggaggtttcacgacctgcaagatacgcttctgaaggcgttcagcgaggacaaggaggagtttaacagaacgacaaccctttcatcttcgtcatcgtcgacgtccactgtagcaaggaacttggcaggtcgaactaggagatgagcaccctggcccattcttgccatattgatagctgattctttttccgtctgaaactgctcgctagatctttcttttctatttaacgagtactattagtgtagcatcatcgtcagatatgatgaagctgttggctttggccctgtaaatcgcctagttatgctcatttttgcttgtatagtaaccgttctccatacagtaatttgttgatggagcttatggatccatgtagtagcagcccttggcctctgactttgatgattggtgagaaacagatggtagaacattaaaaaaaaacagatggtagtttctgctttgcttgattgatctgcatgactgaatactagcactgcccctgtagtttatcactgcccctgtagtttatctctgaagcatctaccgctgaaaaggactactctgtttttaccttgttacagtcgccattttgctgcgccttctgtcctaccagtagtattgccgaagtgaaatttatcttgctggactgaactaccgaggcagagcaaattcaccaccaaaaaccatggagtaaattacatccacggtacgctagggttctcacttcgtcaccgaaagtgtcgaagttcaccaatttaagttgtcaaaatatctcactctaagatcagcattgacttcggaacgcaggcagtagtggtcctcgccgccggaacatcgggggtccgcacgagggcgcctgtcctcgacgttccgcgtccagtggaagtgctcgacgagcggccactccgtggcgctggctggccgcgtccagtggaagttccgtgcccgtgaccggtgaccgtgatgcgccggtgaccgtgggcagccagcgtccagagcagcagcgagagggccacgccgcgtcaggacgccactcccgcgtctataatatcagacacacatacttgacaatcatttcaaatccccaaattatgaattttgattcaaaatttagattcaaattcaaacataacaaatggtccaaattaaaccattcgaagtaaaacacaatacatagaaatttattggtcatgaagcgaccacaaatgctcagctagatcctcgagctc

SEQ ID NO: 2 - protein, 405 aa, Triticum aestivum
MPFFSMQRHLLDFLDKVEVDNLPLILSVANLCNKSCVKLFERCMEMVVRSNLDMITLEKALPQDVIKQITDLRITLGLASPEDNGFPNKHVRRILRALDSDDVELVRMLLTEGQTNLDDAFALHYAVEHCDSKITTELLDIALADVNLRNPRGYTVLHIAAKRRDPKIVVSLLTKGARPSDFTFDGRKAVQISKRLTKHGDYFGNTEEGKPSPNDKLCIEILEQAERRDPQLGEASLSLALAGDCLRGKLLYLENRVALARIMFPIEARVAMDIAQVDGTLEFTLGSSTNPPLEITTVDLNDTSFKMKEEHLARMRALSKTVELGKRFFPRCSNVLDKIMDDEPELASLGRDASSERKRRFHDLQDTLLKAFSEDKEEFNRTTTLSSSSSSTSTVARNLAGRTRR

SEQ ID NO: 3 - DNA, 19 bp, Artificial Sequence
  Description of Artificial Sequence PCR primer KL1
ccattgctactcttgcctc

SEQ ID NO: 4 - DNA, 21 bp, Artificial Sequence
  Description of Artificial Sequence PCR primer KL2
atcgttgtctcccttttaacc

SEQ ID NO: 5 - DNA, 27 bp, Artificial Sequence
  Description of Artificial Sequence PCR primer NIM 2B
ggcaytggaytcwgatgatgttgaryt

SEQ ID NO: 6 - DNA, 31 bp, Artificial Sequence
  Description of Artificial Sequence PCR primer NIM 2D
agttkagcmagdccaactckattttcaarrt

SEQ ID NO: 7 - DNA, 498 bp, Oryza sativa
  CDS (2)..(496)
  misc_feature (2)...(2): Xaa=Leu
ggcaytggattctgatgatgttgagcttgtgaagttgcttcttaacgaatctgagatcaccttggatgatgccaatgcattgcactatgctgctgcttactgtgattcgaaagttgtttcggagttgttagacttgagacttgccaacttgaatttgaagaattcgcgtggatacacggcactccatctggctgctatgaggagagagccagctattatcatgtgtctcctaaacaaaggagcagctgtatcacaattgactgctgatggccagagtgcaatgagtatctgccggaggttaacaaggatgaaagactacaatacaaagatggagcaaggccaagagtcaaacaaagacagattatgtattgatatattagatagggagatgataaggaaacctatggcagtggaagattctgtcacctcgcctttgttggctgacgatcttcacatgaagcttctctaccttgaaaatcgagttggccttgctaaact

SEQ ID NO: 8 - protein, 165 aa, Oryza sativa
  misc_feature (2)...(2): Xaa=Leu
AXDSDDVELVKLLLNESEITLDDANALHYAAAYCDSKVVSELLDLRLANLNLKNSRGYTALHLAAMRREPAIIMCLLNKGAAVSQLTADGQSAMSICRRLTRMKDYNTKMEQGQESNKDRLCIDILDREMIRKPMAVEDSVTSPLLADDLHMKLLYLENRVGLAK

SEQ ID NO: 9 - DNA, 498 bp, Oryza sativa
  CDS (2)..(496)
ggcattggattcagatgatgttgagttagtcaggatgctgctcactgaaggacagacaaatcttgatgatgcgtttgcactgcactacgccgtcgaacattgtgactccaaaattacaaccgagcttttggatctcgcacttgcagatgttaatcatagaaacccaagaggttataccgttcttcacattgctgcgaggcgaagagagcctaaaatcattgtctcccttttaaccaagggggctcggccagcagatgttacattcgatgggagaaaagcggttcaaatctcaaaaagactaacaaaacaaggggattactttggggttaccgaagaaggaaaaccttctccaaaagataggttatgtattgaaatactggagcaagctgaaagaagggacccacaactcggagaagcatcagtttctcttgcaatggcaggtgagagtctacgaggaaggttgctgtaccttgaaaatcgagttggcctggctcaact

SEQ ID NO: 10 - protein, 165 aa, Oryza sativa
ALDSDDVELVRMLLTEGQTNLDDAFALHYAVEHCDSKITTELLDLALADVNHRNPRGYTVLHIAARRREPKIIVSLLTKGARPADVTFDGRKAVQISKRLTKQGDYFGVTEEGKPSPKDRLCIEILEQAERRDPQLGEASVSLAMAGESLRGRLLYLENRVGLAQ

SEQ ID NO: 11 - DNA, 498 bp, Triticum aestivum
  CDS (2)..(496)
ggcactggattcagatgatgttgagcttgtgaagttgcttcttaatgagtctgaaatcaccctagacgacgccaacgcattgcattatgctgcagcttactgcgattctaaagttcttacagagttgttaggcctggaacttgccaacttgaatttgaagaacagtcgtgggtacacagcactccacctagctgctatgaggagagaaccagctattattatgtgtctcttaagcaaaggagcagtggcgtcgcaattgacagatgatggccgccttgcaagtaatatttgtcgaagattaacaagactaaaagattacaatgcaaagatggagcagggccaagagtcaaataaagataggatgtgcattgacatcctagagagggagatgatgaggaatcctatgacagcggaagattcagtcacctcacctttattggctgatgatcttcacatgaaactaagctaccttgaaaatcgagttggccttgctcaact

SEQ ID NO: 12 - protein, 165 aa, Triticum aestivum
ALDSDDVELVKLLLNESEITLDDANALHYAAAYCDSKVLTELLGLELANLNLKNSRGYTALHLAAMRREPAIIMCLLSKGAVASQLTDDGRLASNICRRLTRLKDYNAKMEQGQESNKDRMCIDILEREMMRNPMTAEDSVTSPLLADDLHMKLSYLENRVGLAQ

SEQ ID NO: 13 - DNA, 2326 bp, Oryza sativa
  CDS (419)..(1954)
ggccgcgagccaaagcccctggtttcctcgcaactgcctccccgcgattccgtttgacccccactgttcttctcccctaccaccaccaggtcgccgtcgcttccaatttccaaataattccctccactccggccgctcgcgaggatagaaaaggatttctttttctctctctctctctccccctctctccgagatccgtttcccaaacaggcggggggtcgaaagtgtttggtactttggtttggggagcttgtttgccgacgcggatctgcgtggagacgagcagaggggggagcgccggaattgggtggtttggcccgggaggcgccggaaagtgggggagcctttggattccccgaacccgccatggtgatccggcacgagtagtagtggtggtggtggtattagtagcagtgagatgccggcgcgtagcgcggtggtggtaatagccatggagccctcgtcgtccatcaccatcgcgtcgtcgtcctcgtacctctcgaacgggtctagcccgcggtacaagatggaggagctcgtgccgggaggccgcgtggggcgcgacgccttcctgtcgctgctgggttacctgtacacgggcaagctccggccggcgccggatgacgtggtgtcctgcgccgaccccatgtgcccgcacgactcgtgcccgccggcgatcaggttcaacgtcgagcaaatgtacgcggcgtgggcgttcaagatcaccgagctcatctcgctgttccagcgacggcttcttaacttcgtcgataagactctagtagaagatgttcttccaattctgcaagttgcttttcattcagagctgactccagtgcttgaaaaatgtattcggagaattgcaagatcaaatcttgataatgtatcgttggataaggaacttcctccagaagttgctgttcagataaaagagattcgccaaaaatctcagccaaatgagggtgacaccgtcatttcagaccctgtacatgagaaaagggtcagaagaatccacagggcactggattctgatgatgttgagcttgtgaagttgcttcttaacgaatctgagatcaccttggatgatgccaatgcattgcactatgctgctgcttactgtgattcgaaagttgtttcggagttgttagacttgagacttgccaacttgaatttgaagaattcgcgtggatacacggcactccatctggctgctatgaggagagagccagctattatcatgtgtctcctaaacaaaggagcagctgtatcacaattgactgctgatggccagagtgcaatgagtatctgccggaggttaacaaggatgaaagactacaatacaaagatggagcaaggccaagagtcaaacaaagacagattatgtattgatatattagatagggagatgataaggaaacctatggcagtggaagattctgtcacctcgcctttgttggctgacgatcttcacatgaagcttctctaccttgaaaacagagttgcatttgcaagattattttttcctgcagaagcaaaggttgcaatgcaaattgcacaagcagacaccacaccagaatttggcattgttcctgcagctagcacttctggaaaattgaaggaagtcgatctgaacgagacaccagtaacacaaaacaaaaggctccgttcaagggtggatgcactcatgaaaacagttgagctgggacgtcgctacttccctaactgctcgcaggtgctcgacaaatttctggaggatgatttgcccgatagtcctgatgcactcgacctccaaaatggcacttctgatgagcaaaatgttaaaaggatgcggttctgtgagttaaaggaggatgtgcgcaaggcattcagcaaagacagagctgataatagcatgttttctatcttgtcatcttcatcgtcatcttcgccacctcccaaggttgcaaagaaatgacagaagttttgtaacaaatttccgctcgtgatgttactgggacaagagatatcgatcaatagacctgtatagtcttacagtggtataacaattagatatcgaagcttcttcgaatattagaaagtgctgttctgggctgcactcagctggtttatgggacccatgcggtgaaactggcaaaagaaaaccagctgattagagrctccaaagcagtgtctctcgtgaatatgtttgtagcattctgttttgttcaggatggctataatgataaaatcttttcaatagatatatagctaattgtctcgtaaaaaaaaaaaaaaaaaaaaaaaaaaaaaaaaaaaaaaaaaaaaaaaaaaaaaaaaaaaaaaaaaa

SEQ ID NO: 14 - protein, 511 aa, Oryza sativa
MPARSAVVVIAMEPSSSITIASSSSYLSNGSSPRYKMEELVPGGRVGRDAFLSLLGYLYTGKLRPAPDDVVSCADPMCPHDSCPPAIRFNVEQMYAAWAFKITELISLFQRRLLNFVDKTLVEDVLPILQVAFHSELTPVLEKCIRRIARSNLDNVSLDKELPPEVAVQIKEIRQKSQPNEGDTVISDPVHEKRVRRIHRALDSDDVELVKLLLNESEITLDDANALHYAAAYCDSKVVSELLDLRLANLNLKNSRGYTALHLAAMRREPAIIMCLLNKGAAVSQLTADGQSAMSICRRLTRMKDYNTKMEQGQESNKDRLCIDILDREMIRKPMAVEDSVTSPLLADDLHMKLLYLENRVAFARLFFPAEAKVAMQIAQADTTPEFGIVPAASTSGKLKEVDLNETPVTQNKRLRSRVDALMKTVELGRRYFPNCSQVLDKFLEDDLPDSPDALDLQNGTSDEQNVKRMRFCELKEDVRKAFSKDRADNSMFSILSSSSSSSPPPKVAKK

SEQ ID NO: 15 - DNA, 1565 bp, Oryza sativa
  CDS (1)..(1263)
ggcgccttcccaccagctcgggcgggaggcctcctcctcctcctcctcctcgccgagctcaccaacctcttccagcggcgtctccttgatgtccttgataaggttgaagtagataaccttctattgatcttatctgttgccaacttatgcaacaaatcttgcatgaaactgcttgaaagatgccttgatatggtagtccggtcaaaccttgacatgattactcttgagaagtcattgcctccagatgttatcaagcagattattgatgcacgcctaagcctcggattaatttcaccagaaaacaagggatttcctaacaaacatgtgaggaggatacacagagcccttgactctgacgatgtagagctagtcaggatgctgctcactgaaggacagacaaatcttgatgatgcgtttgcactgcactacgccgtcgaacattgtgactccaaaattacaaccgagcttttggatctcgcacttgcagatgttaatcatagaaacccaagaggttatactgttcttcacattgctgcgaggcgaagagagcctaaaatcattgtctcccttttaaccaagggggctcgaccagcagatgttacattcgatgggagaaaagcggttcaaatctcaaaaagactaacaaaacaaggggattactttggggttaccgaagaaggaaaaccttctccaaaagataggttatgtattgaaatactggagcaagctgaaagaagggacccacaactcggagaagcatcagtttctcttgcaatggcaggtgagagtctacgaggaaggttgctgtatcttgaaaaccgagttgctttggcgaggattatgtttccgatggaggcaagagtagcaatggatattgctcaagtggatggaactttggaatttaacctgggttctggtgcaaatccacctcctgaaagacaacggacaactgttgatctaaatgaaagtcctttcataatgaaagaagaacacttagctcggatgacggcactctccaaaacagtggagctcgggaaacgctttttcccgcgatgttcgaacgtgctcgacaagatcatggatgatgaaactgatccggtttccctcggaagagacacgtccgcggagaagaggaagaggtttcatgacctgcaggatgttcttcagaaggcattccacgaggacaaggaggagaatgacaggtcggggctctcgtcgtcgtcgtcatcgacatcgatcggggccattcgaccaaggagatgaacaccattgctcccaaatagttgccatattgatagctaactgtcctcctggagctactcacctgatggttgccttctgtcaattgccccccaaatatattctcaatggtttaggcttgtacagtattagttcttacagctattgccccgtcaattgtgaaacgcagaagtttcactagtgcttgtactcgaggtgtaatacaagtgcttgaattttgagttgtacttggaatttccaggtggtttgctcgtaaaaatgagatgatttcttggctcccaaaaaaaaaaaaaaaaaaaaaaaaa

SEQ ID NO: 16 - protein, 420 aa, Oryza sativa
GAFPPARAGGLLLLLLLAELTNLFQRRLLDVLDKVEVDNLLLILSVANLCNKSCMKLLERCLDMVVRSNLDMITLEKSLPPDVIKQIIDARLSLGLISPENKGFPNKHVRRIHRALDSDDVELVRMLLTEGQTNLDDAFALHYAVEHCDSKITTELLDLALADVNHRNPRGYTVLHIAARRREPKIIVSLLTKGARPADVTFDGRKAVQISKRLTKQGDYFGVTEEGKPSPKDRLCIEILEQAERRDPQLGEASVSLAMAGESLRGRLLYLENRVALARIMFPMEARVAMDIAQVDGTLEFNLGSGANPPPERQRTTVDLNESPFIMKEEHLARMTALSKTVELGKRFFPRCSNVLDKIMDDETDPVSLGRDTSAEKRKRFHDLQDVLQKAFHEDKEENDRSGLSSSSSSTSIGAIRPRR

SEQ ID NO: 17 - DNA, 2446 bp, Triticum aestivum
  CDS (148)..(1977)
  misc_feature (2368): n = a, t, c, or g
ggccgacgcggccctgcgtggtgccaaacaaagcctgcccgaattgcgcagttcggccgggagagaccaaaaggcagcctcccccctttgccttcacacatggtggtccggctctagggccctttcgcctcgtgcttggcggcggtgatggagccgtcgtcgtccatcacgttcgcctcctcgtcgtcctacctgtccaacggctccagcccctgctccgtcgctctggcgccactgcccgcggcggacgggtggggagggggtggtggagggggagggagcagcagcagcgtcgaggctgtgagcctgaatcgcctcagcaacaacctcgagcgcctcctcctcgattctgaactcgactgcagcgacgccgacgtcgacatggcggacggcgggccgcccatccccgtccaccgctgcatcctcgccgcgcgcagccccttcttccacgacctcttccgcgcccgcgggagccgcagtgatggggcagtcaccgcctccgcctccgcctccgccaccagtggcggagcgggaggggatgtgaccgggaggccgcagtacaagatggaggacctcgtcccaggtggccgtgttggtcgcgaggccttcctggcgttcatggggtacctctacaccggcaggctccggcccgcgccactggacgtggtgtcatgtgctgatcttgtgtgcccgcacgactcgtgcccaccggccatcaggttcgccgtcgagctcatgtacgcggcgtggaccttcaggatccccgagctcatgtcgctgttccagcgacggcttatgaactttatcgacaagactctagctgaagacgtcctgcctatcttgcaagttgccttccactcagagcttactcaagtgcgtggaaaatgtgttcaaaggattgcaagatcagatcttgatattatgtctttggataaggaactccctccagaaattgctgatgagataaaaaaaatccgacagaaatcttccccaattgatggtgacaccatcatttcggaccctgttcacgagaaaagagtaagaagaatccacagggcactggattctgatgatgttgaacttgtgaagttgcttcttaatgagtctgaaatcaccctagacgacgccaacgcattgcattatgctgcagcttactgcgattctaaagttcttacagagttgttaggcctggaacttgccaacttgaatttgaagaacagtcgtgggtacacagcactccacctagctgctatgaggagagaaccagctattattatgtgtctcttaagcaaaggagcagtggcgtcgcaattgacagatgatggccgccttgcaagtaatatttgtcgaagattaacaagactaaaagattacaatgcaaagatggagcagggccaagagtcaaataaagataggatgtgcattgacatcctagagagggagatgatgaggaatcctatgacagcggaagattcagtcacctcacctttattggctgatgatcttcacatgaaactaagctacctggaaaatagagtcgcgtttgcaagactattcttccctgctgaagcgaaggttgcgatgcaaattgcgcaagcagacatcacaccagaagttggtggtttttctgcagcaagtacttctggtaaactgagggaagtcgatctgaatgagacgccagtaacaaaaaacaaaaggctacgttcgagggtggatgcactagtgaaaacagtggaactgggccgtcggtacttcccaaactgctcgcaggtgctcgacaaattcttggaagatggcctgcctgatggccttgatgcattccagcagcaaagcggcacccctgatgagcaacaggtgaagaagatgcgcttctgcgaggtgaaggaggacgtgcgcaaagcatacagcaaagacacggccgataacagcatgttttcagccctgtcgtcaaactcctcatcctcggcgatgaagtgaaggtactgtaacaggctgttttctggagatgtcaggactaaagagggatcgctggtcatgcgcatgtatagtgctcmccatcgtgtaaaactgaatatgaacatgaaagaaggccccaaaatagtagaagatgatatatactttgctggacttggagtttgttggagaaggctgtgccatcccattccagattcccaatatcaattttcccatgctggttgtgaagacagagccgcggatcatccagctccgacgctatgcatgcgtgcagcctgctgtatttgtttcgcatagctgcaatacttatatgtttaataatagtactagggagtagtaggttattgaggctgtagcggaagttggaacctmccttaatgtaagtgaaaggggncagttgcccwttgtcgaattgttgttatcaatacatagttgattttcgbmaaaaaaaaaaaaaaaaaaaaaaaaaaaa

SEQ ID NO: 18 - protein, 609 aa, Triticum aestivum
MEPSSSITFASSSSYLSNGSSPCSVALAPLPAADGWGGGGGGGGSSSSVEAVSLNRLSNNLERLLLDSELDCSDADVDMADGGPPIPVHRCILAARSPFFHDLFRARGSRSDGAVTASASASATSGGAGGDVTGRPQYKMEDLVPGGRVGREAFLAFMGYLYTGRLRPAPLDVVSCADLVCPHDSCPPAIRFAVELMYAAWTFRIPELMSLFQRRLMNFIDKTLAEDVLPILQVAFHSELTQVRGKCVQRIARSDLDIMSLDKELPPEIADEIKKIRQKSSPIDGDTIISDPVHEKRVRRIHRALDSDDVELVKLLLNESEITLDDANALHYAAAYCDSKVLTELLGLELANLNLKNSRGYTALHLAAMRREPAIIMCLLSKGAVASQLTDDGRLASNICRRLTRLKDYNAKMEQGQESNKDRMCIDILEREMMRNPMTAEDSVTSPLLADDLHMKLSYLENRVAFARLFFPAEAKVAMQIAQADITPEVGGFSAASTSGKLREVDLNETPVTKNKRLRSRVDALVKTVELGRRYFPNCSQVLDKFLEDGLPDGLDAFQQQSGTPDEQQVKKMRFCEVKEDVRKAYSKDTADNSMFSALSSNSSSSAMK

SEQ ID NO: 19 - DNA, 1668 bp, Triticum aestivum
  CDS (451)..(1668)
tgtacttgcgagcatttgaaacacataaaattacttttgataggttacttaaatatatgcaacttcgatgcagaggctggggtaataaaatcttccattttctattttttgaaatacttgttgacagggctgtaatcaaattgggttaatcaatgtatgtgtttgtattcttaaaatattacttatcagattagaccgtttatgcgtctatattcttatcaatccgtatggctgtgtcgagacttcggatttttatgtattttttagtgatgatatgcttttccttcttagctttgtcatactgagatttgtgttttaataattctgacttcgctgcagatgatttgcccgtgtatcgtttgatgctaactctcgtcgacttgctacttgtaacagttctctattgttctattgtttcatgtttttgagaagcgagtactaacccatgttatgcccttcttttccatgcagcggcatctccttgatttccttgataaagttgaagtggataaccttccgttgatcttatctgttgcaaacttatgcaacaaatcttgcgtgaaactgttcgagagatgcatggagatggtagtccggtcaaatcttgacatgattactctagagaaagcattgcctcaagatgtcatcaagcaaattactgatttacggataactcttggattagcttcacccgaagacaatggctttcctaacaaacacgtaagaaggatactcagagcacttgattctgatgatgtggagcttgtcaggatgctgctcacagaagggcagactaaccttgatgatgcatttgcattgcactatgctgtagaacactgtgactcaaaaattacaacagaacttctggacatcgcacttgcggatgttaatctcagaaacccaagaggttatactgttcttcacatcgccgctaagcggagagatcctaaaatcgttgtctcccttttaaccaaaggtgcccggccttcagattttacatttgatggaagaaaagcagttcaaatctcaaagagactcacaaaacatggtgattattttgggaatactgaagaaggaaagccgtctcccaatgataaattatgcattgagatattggagcaagctgaaagaagggatccacaacttggagaagcatcactttctcttgcattggctggtgactgtcttcgtggaaagttactgtaccttgaaaaccgagttgctttggcaaggataatgtttccaattgaggcaagagtagcaatggacattgctcaagtggatggtactttggaatttacccttggttctagtacaaatccacctctggagataacaaccgttgatctgaatgatacttctttcaaaatgaaggaggaacacttagctcggatgagagccctctccaaaacagttgaactcggcaaacgtttcttcccacgctgttcaaatgtgctggacaagatcatggacgatgaacctgagctggcttccctcggaagagatgcatcctccgagaggaagaggaggtttcacgacctgcaagatacgcttctgaaggcgttcagcgaggacaaggaggagtttaacagaacgacaaccctttcatcttcgtcatcgtcgacgtccactgtagcaaggaacttggcaggtcgaactaggagatga

SEQ ID NO: 20 - protein, 405 aa, Triticum aestivum; identical to SEQ ID NO: 2